SEQUENCE LISTING (34 sequences)

SEQ ID NO: 1 - DNA, 112 bp, Homo sapiens
gcttcccgcccggagcccaccctgggaagagccgcgcgcgttcttgcgtctgccccgactgcggggacaatggaagcgtcctcagggcagagtccagaccccgtgtgtggtt

SEQ ID NO: 2 - DNA, 112 bp, Artificial sequence
  Synthetic
gtttttcgttcggagtttattttgggaagagtcgcgcgcgtttttgcgtttgtttcgattgcggggataatggaagcgtttttagggtagagtttagatttcgtgtgtggtt

SEQ ID NO: 3 - DNA, 23 bp, Artificial sequence
  Synthetic
agtttattttgggaagagtcgcg

SEQ ID NO: 4 - DNA, 20 bp, Artificial sequence
  Synthetic
ccattatccccgcaatcgaa

SEQ ID NO: 5 - DNA, 25 bp, Artificial sequence
  Synthetic
cgcgccgagggcgcgtttttgcgtt

SEQ ID NO: 6 - DNA, 72 bp, Homo sapiens
gggaccggagccgagcctagcgcggcgcccgcgacccgtcagccgcggctcctgctccctcgatcccgcgcg

SEQ ID NO: 7 - DNA, 72 bp, Artificial sequence
  Synthetic
gggatcggagtcgagtttagcgcggcgttcgcgattcgttagtcgcggtttttgttttttcgatttcgcgcg

SEQ ID NO: 8 - DNA, 19 bp, Artificial sequence
  Synthetic
tcggagtcgagtttagcgc

SEQ ID NO: 9 - DNA, 24 bp, Artificial sequence
  Synthetic
cgaaatcgaaaaaacaaaaaccgc

SEQ ID NO: 10 - DNA, 20 bp, Artificial sequence
  Synthetic
cgccgaggcggcgttcgcga

SEQ ID NO: 11 - DNA, 21 bp, Artificial sequence
  Synthetic
ccacggacgcggcgttcgcga

SEQ ID NO: 12 - DNA, 111 bp, Homo sapiens
gaccggggtgtcccccaggctccgcgtcgcggtccccgctcgccctcccgcccgcccaccgggcaccccagccgcgcagaaggcggaagccacgcgcgagggaccgcggtc

SEQ ID NO: 13 - DNA, 111 bp, Artificial sequence
  Synthetic
gatcggggtgttttttaggtttcgcgtcgcggttttcgttcgttttttcgttcgtttatcgggtattttagtcgcgtagaaggcggaagttacgcgcgagggatcgcggtt

SEQ ID NO: 14 - DNA, 21 bp, Artificial sequence
  Synthetic
cggttttcgttcgttttttcg

SEQ ID NO: 15 - DNA, 18 bp, Artificial sequence
  Synthetic
ccgccttctacgcgacta

SEQ ID NO: 16 - DNA, 21 bp, Artificial sequence
  Synthetic
ccacggacggttcgtttatcg

SEQ ID NO: 17 - DNA, 70 bp, Homo sapiens
ggggccggggccgacagcccacgctggcgcggcaggcgcgtgcgcccgccgttttcgtgagcccgagcag

SEQ ID NO: 18 - DNA, 70 bp, Artificial sequence
  Synthetic
ggggtcggggtcgatagtttacgttggcgcggtaggcgcgtgcgttcgtcgttttcgtgagttcgagtag

SEQ ID NO: 19 - DNA, 20 bp, Artificial sequence
  Synthetic
gtcggggtcgatagtttacg

SEQ ID NO: 20 - DNA, 19 bp, Artificial sequence
  Synthetic
actcgaactcacgaaaacg

SEQ ID NO: 21 - DNA, 21 bp, Artificial sequence
  Synthetic
ccacggacggacgaacgcacg

SEQ ID NO: 22 - DNA, 139 bp, Homo sapiens
ggccacacaggcccactctggccctctgagcccccggcggacccagggcattcaaggagcggctctgggctgccagcgcaggcctccgcgcaaacacagcaggctggaagtggcgctcatcaccggcacgtcttcccag

SEQ ID NO: 23 - DNA, 139 bp, Artificial sequence
  Synthetic
ggttatataggtttattttggttttttgagttttcggcggatttagggtatttaaggagcggttttgggttgttagcgtaggttttcgcgtaaatatagtaggttggaagtggcgtttattatcggtacgtttttttag

SEQ ID NO: 24 - DNA, 27 bp, Artificial sequence
  Synthetic
ggtttattttggttttttgagttttcg

SEQ ID NO: 25 - DNA, 24 bp, Artificial sequence
  Synthetic
tccaacctactatatttacgcgaa

SEQ ID NO: 26 - DNA, 21 bp, Artificial sequence
  Synthetic
ccacggacggcggatttaggg

SEQ ID NO: 27 - DNA, 224 bp, Homo sapiens
ctctgacctgagtctcctttggaactctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaaga

SEQ ID NO: 28 - DNA, 19 bp, Artificial sequence
  Synthetic
ccatgaggctggtgtaaag

SEQ ID NO: 29 - DNA, 23 bp, Artificial sequence
  Synthetic
tactgtgcacctacttaatacac

SEQ ID NO: 30 - DNA, 20 bp, Artificial sequence
  Synthetic
cgccgagggcggccttggag

SEQ ID NO: 31 - DNA, 104 bp, Artificial sequence
  Synthetic
tggtgtttgtttttttgattaggtgtttaagatagtgttgtgggtgtaggtattaatattggtttgtgtgataaggttatgaggttggtgtaaagcggttttgg

SEQ ID NO: 32 - DNA, 30 bp, Artificial sequence
  Synthetic
gtgtttgtttttttgattaggtgtttaaga

SEQ ID NO: 33 - DNA, 26 bp, Artificial sequence
  Synthetic
ctttacaccaacctcataaccttatc

SEQ ID NO: 34 - DNA, 21 bp, Artificial sequence
  Synthetic
gacgcggagatagtgttgtgg

(19) United States
(12) Patent Application Publication
(10) Pub. No.: US 2023/0190243 A1
(43) Pub. Date: Jun. 22, 2023

(54) INGESTIBLE SAMPLING DEVICE

(71) Applicants: Exact Sciences Corporation, Madison, WI (US); Mayo Foundation for Medical Education and Research, Rochester, MN (US)

(72) Inventors: Robert F. Eisele, Carlsbad, CA (US); Michael E. Williams, San Diego, CA (US); Prasad G. Iyer, Rochester, MN (US); Paul Marrione, Middleton, WI (US); Hatim T. Allawi, Middleton, WI (US); Graham P. Lidgard, Madison, WI (US); Maria Giakoumopoulos, Middleton, WI (US); William R. Taylor, Lake City, MN (US); Tracy C. Yab, Rochester, MN (US); Douglas W. Mahoney, Rochester, MN (US); John B. Kisiel, Rochester, MN (US); David A. Ahlquist, Rochester, MN (US)

(21) Appl. No.: 17/996,032

(22) PCT Filed: Apr. 16, 2021
(86) PCT No.: PCT/US2021/027770

§ 371 Date: Oct. 12, 2022

Related U.S. Application Data
(60) Provisional application No. 63/011,684, filed on Apr. 17, 2020.

(51) Int. Cl. A61B 10/02; A61B 10/00; C12Q 1/6883

(52) U.S. Cl. CPC A61B 10/02; A61B 10/0096; C12Q 1/6883; A61B 2010/0061; C12Q 2600/156

(57) ABSTRACT

The invention relates to cell sampling devices. In particular, the present invention relates to ingestible cell sampling devices for sampling cells in a subject, and methods of use for detecting abnormalities in a subject using the same.

The present application claims priority to U.S. Provisional Application Ser. No. 63/011,684, filed Apr. 17, 2020, which is incorporated herein by reference.

### SEQUENCE LISTING

The text of the computer readable sequence listing filed herewith, titled “38145-601_SEQUENCE_LISTING_ST25”, created Apr. 16, 2021, having a file size of 7,473 bytes, is hereby incorporated by reference in its entirety.

### FIELD OF THE INVENTION

The invention relates to cell sampling devices. In particular, the present invention relates to ingestible cell sampling devices for sampling cells in a subject, and methods of use for detecting abnormalities in a subject using the same.

### BACKGROUND OF THE INVENTION

To diagnose certain diseases of the gastrointestinal tract, an ingestible cell sampling device may be used to collect cells from the surface of the gastrointestinal tract of a patient. However, various issues exist with the cell sampling devices currently used, including difficulty or unpleasantness in swallowing and retrieving the device, detachment of the sponge from the string during use, and/or laceration to the esophagus of the patient upon withdrawal of the device. Accordingly, what is needed are improved ingestible cell sampling devices for use in a subject.

### SUMMARY OF THE INVENTION

In some aspects, provided herein are ingestible cell sampling devices. The device comprises an abrasive sponge housed within a dissolvable capsule, a molded cap, and a string attached to the molded cap. In some embodiments, the abrasive sponge comprises reticulated foam. In some embodiments, the abrasive sponge is compressible. The sponge may be retained in a compressed state by the dissolvable capsule. In some embodiments, the abrasive sponge, when in an uncompressed state, has a shape configured to maximize an exterior dimension while minimizing the total amount of sponge to be contained in a capsule. For example, in some embodiments, the abrasive sponge is formed to have concavities or indentations, and/or to have a void space, as may be provided by removing at least one portion of the abrasive sponge prior to compressing the sponge into the dissolvable capsule, e.g., from inside the sponge, and/or from the top, bottom, and/or side of the abrasive sponge.

In some embodiments, the dissolvable capsule comprises one or more openings, such that a portion of the abrasive sponge is exposed to the external environment at the one or more openings. In some embodiments, the dissolvable capsule comprises a first closed end and a second closed end. In alternative embodiments, the dissolvable capsule comprises a first closed end and a second open end.

In some embodiments, the molded cap comprises an internal surface in contact with an external surface of one end of the capsule and an exterior surface in contact with the external environment. In some embodiments, the molded cap comprises an internal surface in contact with the abrasive sponge and an exterior surface in contact with the external environment. In some embodiments, the molded cap comprises an interior surface in contact with the abrasive sponge and an exterior surface. The exterior surface of the molded cap may be in contact with an internal surface of one end of the capsule. In some embodiments, the interior surface of the molded cap is attached to the abrasive sponge by an adhesive, which is preferably dissolvable.

In some embodiments, the string is attached to the molded cap by a knot. In some embodiments, the string is attached to the molded cap by an adhesive. In some embodiments, the string is attached to the molded cap by a knot and adhesive. The string may comprise a suture. In some embodiments, the string passes through a portion of the abrasive sponge.

In some aspects, provided herein are methods of obtaining a cell sample from a subject. In some embodiments, a method of obtaining a cell sample from a subject comprises providing the ingestible cell sampling device described herein to the subject, and removing all or a portion of the ingestible cell sampling device from the subject. The ingestible cell sampling device may be removed from the subject within 10 minutes of providing the ingestible cell sampling device to the subject.

In some embodiments, the technology provides an ingestible cell sampling device and systems comprising such a device, e.g., for conducting a cell sampling method using a device as described herein.

Embodiments of the technology include:

1. An ingestible cell sampling device comprising:
i) an abrasive sponge housed within a dissolvable capsule, the dissolvable capsule comprising an exterior surface exposed to an external environment;
ii) a molded cap; and
iii) a string having a first end attached to the molded cap.

2. The ingestible cell sampling device of embodiment 1, further comprising a handle, preferably an unswallowable handle, attached to the string.

3. The ingestible cell sampling device of embodiment 1 or 2, wherein the abrasive sponge comprises reticulated foam.

4. The ingestible cell sampling device of any of the preceding embodiments, wherein the abrasive sponge is compressible.

5. The ingestible cell sampling device of embodiment 4, wherein the abrasive sponge is retained in a compressed state by the dissolvable capsule.

6. The ingestible cell sampling device of any of the preceding embodiments, wherein in an uncompressed state, the abrasive sponge comprises at least one void space.

7. The ingestible cell sampling device of embodiment 6, wherein the string passes through at least one void space, preferably at least one concavity.

8. The ingestible cell sampling device of any of the preceding embodiments, wherein the dissolvable capsule comprises one or more openings, wherein a portion of the abrasive sponge is exposed to the external environment at the one or more openings.

9. The ingestible cell sampling device of any of the preceding embodiments, wherein the dissolvable capsule comprises a first end and a second end, wherein:
a) the first end is closed and the second end is closed; or
b) the first end is closed and the second end is open.

10. The ingestible cell sampling device of embodiment 9, wherein the molded cap comprises a cap interior surface and a cap exterior surface, wherein the cap interior surface is in contact with the exterior surface of the capsule at the first closed end, and the cap exterior surface is in contact with the external environment.

11. The ingestible cell sampling device of embodiment 9, wherein the molded cap comprises a cap interior surface and a cap exterior surface, wherein the cap interior surface is in contact with the abrasive sponge.

12. The ingestible cell sampling device of embodiment 11, wherein the cap exterior surface is in contact with an internal surface of the capsule at the first closed end.

13. The ingestible cell sampling device of embodiment 11 or 12, wherein the cap interior surface is attached to the abrasive sponge by an adhesive.

14. The ingestible cell sampling device of embodiment 9, wherein the molded cap comprises a cap interior surface in contact with the abrasive sponge and a cap exterior surface in contact with the external environment.

15. The ingestible cell sampling device of embodiment 14, wherein the cap interior surface is attached to the abrasive sponge.

16. The ingestible cell sampling device of embodiment 15, wherein the cap interior surface is attached to the abrasive sponge by an adhesive.

17. The ingestible cell sampling device of any of the preceding embodiments, wherein the string is attached to the molded cap by a knot and/or an adhesive.

18. The ingestible cell sample device of any of the proceeding embodiments, wherein the string has one or more calibration markings.

19. The ingestible cell sampling device of any of the preceding embodiments, wherein the string comprises a suture.

20. The ingestible cell sampling device of any of the preceding embodiments, wherein the string passes through a portion of the abrasive sponge.

21. The ingestible cell sampling device of any of the preceding embodiments, wherein the molded cap comprises a button.

22. A system or kit for obtaining a cell sample from a subject, comprising an ingestible cell sampling device of any of the preceding embodiments; and further comprising one or more of:
i) a container to receive an abrasive sponge comprising collected cells;
ii) a cell preservative reagent, preferably a buffer reagent;
iii) a microscope slide;
iv) an assay plate;
v) a local anesthetic treatment, preferably a local anaesthetic spray;
vi) a component of a drinkable solution; preferably a pre-mixed drinkable solution;

and
vii) a lubricant, preferably a lubricant gel or liquid.

23. A method of obtaining a cell sample from a subject, comprising:
i) orally administering an abrasive sponge housed within a dissolvable capsule of a ingestible cell sampling device of any one of embodiments 1-21 to the subject, and
ii) withdrawing from the subject the abrasive sponge, wherein the abrasive sponge collects a cell sample from the subject during the withdrawing.

24. The method of embodiment 23, wherein the withdrawing is within 10 minutes of the orally administering.

25. The method of embodiment 23 or embodiment 24, wherein during the orally administering, the subject swallows the dissolvable capsule of the ingestible cell sampling device.

26. A method of characterizing a cell sample collected according to any one of embodiments 23-25, comprising assaying the cell sample for at least one biomarker.

27. The method of embodiment 26, wherein the at least one biomarker is selected comprises one or more of a protein and a nucleic acid.

28. The method of embodiment 26 or embodiment 27, wherein the at least one biomarker comprises DNA comprising at least a portion of a gene selected from the group consisting of NDRG4, ZNF682, VAV3, BMP3, ZNF568, FER1L4, ANKRD13B, CD1D, CDKN2A , CHST2, CNNM1, DIO3, DOCK2, DTX1, ELMO1, FERMT3, FLI1, GRIN2D, HUNK , JAM3, LRRC4, OPLAH, PDGFD, PKIA, PPP2R5C, QKI, SEP9, SFMBT2, SLC12A8, TBX15, TSPYL5, ZNF304, and ZNF671.

29. The method of embodiment 28, wherein assaying the at least one biomarker comprises determining the DNA to determine the methylation state of the gene.

30. The method of any one of embodiments 26-29, wherein assaying the at least one biomarker comprises assaying 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 of the biomarkers from the group consisting of NDRG4, ZNF682, VAV3, BMP3, ZNF568, FER1L4, ANKRD13B, CD1D, CDKN2A , CHST2, CNNM1, DIO3, DOCK2, DTX1, ELMO1, FERMT3, FLI1, GRIN2D, HUNK , JAM3, LRRC4, OPLAH, PDGFD, PKIA, PPP2R5C, QKI, SEP9, SFMBT2, SLC12A8, TBX15, TSPYL5, ZNF304, and ZNF671.

31. The method of embodiment 30, wherein assaying the at least one biomarker comprises assaying the methylation state of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 genes from the group consisting of NDRG4, ZNF682, VAV3, BMP3, ZNF568, FER1L4, ANKRD13B, CD1D, CDKN2A , CHST2, CNNM1, DIO3, DOCK2, DTX1, ELMO1, FERMT3, FLI1, GRIN2D, HUNK , JAM3, LRRC4, OPLAH, PDGFD, PKIA, PPP2R5C, QK1, SEP9, SFMBT2, SLC12A8, TBX15, TSPYL5, ZNF304, and ZNF671.

31. The method of any one of embodiments 28-30, wherein assaying the at least one biomarker comprises assaying the methylation state of at least one gene selected from the group consisting of: ANKRD13B, CHST2, CNNM1, DOCK2, DTX1, FER1L4, FERMT3, FLI1, GRIN2D, JAM3, LRRC4, OPLAH, PDGFD, PKIA, PPP2R5C, QK1, SEP9, SFMBT2, SLC12A8, TBX15, TSPYL5, VAV3, ZNF304, ZNF568, and ZNF671.

32. The method of any one of embodiments 28-30, wherein assaying the at least one biomarker comprises assaying the methylation state of at least one gene selected from the group consisting of: BMP3, NDRG4, VAV3, SFMBT2, DIO3, HUNK, ELMO1, CD1D, CDKN2A, and OPLAH.

33. The method of any one of embodiments 28-30, wherein assaying the at least one biomarker comprises assaying the methylation state of at least one gene selected from the group consisting of NDRG4, ZNF682, VAV3, BMP3, ZNF568, and FER1L4.

34. The method of embodiment 33, comprising assaying the methylation state of the group of genes consisting of NDRG4, ZNF682, VAV3, BMP3, ZNF568, and FER1L4.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “abrasive” as used herein refers to a material capable of removing cells from a surface, and preferably collecting the cells removed from the surface. For example, abrasive may indicate a material capable of removing cells from the esophagus of a subject. Preferably, an abrasive material is capable of removing cells from a surface (e.g., the esophagus) without causing damage to the esophagus.

The term “dissolvable” as used herein refers to a material capable of dissolving when exposed to the environment within the stomach cavity.

The term “esophageal disorder” refers to types of disorder associated with the esophagus and/or esophageal tissue. Examples of esophageal disorders include, but are not limited to, Barrett's esophagus (BE), Barrett's esophageal dysplasia (BED), Barrett's esophageal low-grade dysplasia (BE-LGD), Barrett's esophageal high-grade dysplasia (BE-HGD), and esophageal adenocarcinoma (EAC).

The “ingestible cell sampling device” of the technology comprises an ingestible portion, e.g., an “ingestible assembly” comprising an abrasive sponge housed within a dissolvable capsule and attached to a string; and a non-ingestible portion, e.g., a portion of the string that is not ingested during use, which is preferably attached to a handle.

The term “handle” as used herein in reference to a non-swallowable component of an ingestible sampling device suitable for a user or a third party to hold, e.g., oral administration of the ingestible assembly of the ingestible cell sampling device.

The term “molded” as used herein means any suitable means of fabricating a component, e.g., a cap or capsule, including but not limited to injection molding, compression molding, transfer molding, sintering, various means of 3-D additive printing, stereolithography, and machining.

The term “capsule” as used herein refers to any component or material serving to surround or encase a sponge, preferably in a manner that renders the sponge swallowable. In some embodiments, the capsule is dissolvable. A capsule encompasses any material or device that encloses a sponge, preferably a sponge in a compressed state, and that provides an surface suitable for swallowing, e.g., a surface of sufficient smoothness and/or slickness to facilitate swallowing of the sponge. A capsule may be formed separately, e.g., as a molded empty container in which a sponge is later partially or completely enclosed, or a capsule may be formed as part of process of encasing the sponge, e.g., as a coating, wrapping, or other binding treatment applied during or after compression of the sponge, and having the effect of holding the enclosed sponge in the compressed state.

The term “cap” as used herein in reference to an ingestible sampling device refers to a rigid or semi-rigid component attached or attachable at or near an end of a string of the ingestible sampling device, preferably comprising one or more holes or openings for attachment of the string, e.g., with a loop or knot. A cap component of the ingestible assembly is shaped to be suitable for swallowing, e.g., having a cupped shape that follow an external contour of a a capsule, or being shaped to fit within a capsule.

The term “button” as used herein in reference to an ingestible sampling device refers to a rigid or semi-rigid component attached or attachable at or near an end of a string of the ingestible sampling device, preferably comprising one or more holes or openings for attachment of the string, e.g., with a loop or knot. The term “button” is an example of a molded cap having a disk-like shape. In some embodiments, a button is sized to fit within the dissolvable capsule of the ingestible sampling device.

The term “assaying” as used herein in reference to cell samples refers to qualitatively assessing or quantitatively measuring an aspect of a sample, e.g., assessing or measuring the presence, amount, state, or functionality of a target entity, e.g., a biomarker.

The term “string” as used herein refers broadly to any cord, thread, filament, cable, strand, fiber, ribbon, webbing, suture, lacing, or other flexible tethering material, including materials that are a single filament, and that comprise multiple filaments, e.g., comprising one or more strands that are, for example, twisted, braided, woven, fused, or otherwise combined to form a string, and may comprise strands or filaments of the same or different natural, synthetic, or hybrid material, e.g., silk, cotton, polyester, nylon, polypropylene, cellulose, and the like. A string or individual filaments of a string may be solid, e.g., a single cord or filament of a flexible material such as nylon or polypropylene, or a string may comprise one or more hollow strands, e.g., a tubing of nylon, polypropylene, or other flexible material. A string may comprise one or more informative markings, e.g., calibration markings indicating, for example, a distance between a marking and an end of the string. A series of calibration markings may be provided along the string, and may be at evenly spaced intervals, or at intervals of different lengths.

The term “reticulated” as used herein refers to a porous, low density material, e.g., a foam. Reticulated material comprises open pores or cells, with few intact closed cells.

The term “compressible” as used herein in reference to components of an ingestible sampling device, e.g., an abrasive sponge, refers to a material that is capable of being reversibly forced or pressed into a smaller space or narrower compass in at least one dimension, and that is capable of expanding to an uncompressed dimension when the compressing force is removed.

As used herein, the terms “biomarker” and “marker” are used interchangeably, and refer to a biological material (e.g., a nucleic acid, or a region of a nucleic acid, or a protein) that may be used to distinguish non-normal cells (e.g., cancer cells) from normal cells, e.g., based on presence, absence, or status (e.g., methylation state or mutation) of the marker substance. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid or protein that are biomarkers may be referred to, e.g., as “marker genes,” “marker regions,” “marker sequences,” “marker loci,” etc.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human.

The term “sample” is used in its broadest sense. In one sense it can refer to an animal cell or tissue. In another sense, it refers to a specimen or culture obtained from any source, as well as biological and environmental samples. Biological samples encompass fluids, solids, tissues, and gases. Environmental samples include environmental material such as surface matter, soil, water, and industrial samples. These examples are not to be construed as limiting the sample types applicable to the present invention.

As used herein, the term “cell sample” refers to a sample that comprises a cell (e.g., an intact cell from a subject) or cellular material (e.g., material from cells from the subject that are not intact cells).

As used herein, a “methylation state”, “methylation profile”, and “methylation status” of a nucleic acid molecule refers to the presence of absence of one or more methylated nucleobases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is generally considered unmethylated. The methylation state of a particular nucleic acid sequence (e.g., a gene marker or region of a gene marker) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles.

As used herein, the term “nucleic acid detection assay” refers to any method of determining the nucleotide composition of a nucleic acid of interest. Nucleic acid detection assays include but are not limited to, DNA sequencing methods, probe hybridization methods, structure specific cleavage assays (e.g., the INVADER assay, (Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and US 2009/0253142, each of which is herein incorporated by reference in its entirety for all purposes); enzyme mismatch cleavage methods (e.g., U.S. Pat. Nos. 6,110,684, 5,958,692, 5,851,770, herein incorporated by reference in their entireties); polymerase chain reaction (PCR) (including real-time PCR, such as Taqman PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Baranay Proc. Natl. Acad. Sci USA 88, 189-93 (1991)); and sandwich hybridization methods (e.g., U.S. Pat. No. 5,288,609, herein incorporated by reference in its entirety). Additional methods are described in U.S. patent application Ser. No. 15/881,409 of Allawi, et al., filed Jan. 26, 2018, incorporated herein by reference in its entirety.

In some embodiments, target nucleic acid is amplified (e.g., by polymerase chain reaction, e.g., as described by K. B. Mullis in U.S. Pat. Nos. 4,683,195, 4,683,202, and 4,965,188) and amplified nucleic acid is detected simultaneously using an invasive cleavage assay. Assays configured for performing a detection assay (e.g., invasive cleavage assay) in combination with an amplification assay are described in U.S. Pat. No. 9,096,893, incorporated herein by reference in its entirety for all purposes. Additional amplification plus invasive cleavage detection configurations, termed the QuARTS method, are described in, e.g., in U.S. Pat. Nos. 8,361,720; 8,715,937; 8,916,344; and 9,212,392, each of which is incorporated herein by reference for all purposes. Additional modified QUARTS methods, termed LQAS AND TELQAS, are described in, e.g., U.S. Patent Publication No. US20200248233A1, U.S. Pat. No. 10,648,025, International Application Publication No. WO20211040726A1, and International Application publication No. WO2020206256A1.

The term “invasive cleavage structure” as used herein refers to a cleavage structure comprising i) a target nucleic acid, ii) an upstream nucleic acid (e.g., an invasive or “INVADER” oligonucleotide), and iii) a downstream nucleic acid (e.g., a probe), where the upstream and downstream nucleic acids anneal to contiguous regions of the target nucleic acid, and where an overlap forms between the a 3′ portion of the upstream nucleic acid and duplex formed between the downstream nucleic acid and the target nucleic acid. An overlap occurs where one or more bases from the upstream and downstream nucleic acids occupy the same position with respect to a target nucleic acid base, whether or not the overlapping base(s) of the upstream nucleic acid are complementary with the target nucleic acid, and whether or not those bases are natural bases or non-natural bases. In some embodiments, the 3′ portion of the upstream nucleic acid that overlaps with the downstream duplex is a non-base chemical moiety such as an aromatic ring structure, e.g., as disclosed, for example, in U.S. Pat. No. 6,090,543, incorporated herein by reference in its entirety. In some embodiments, one or more of the nucleic acids may be attached to each other, e.g., through a covalent linkage such as nucleic acid stem-loop, or through a non-nucleic acid chemical linkage (e.g., a multi-carbon chain). As used herein, the term “flap endonuclease assay” includes “INVADER” invasive cleavage assays, QuARTS assays, LQAS and TELQAS assays, as described above.

A “flap oligonucleotide” refers to an oligonucleotide cleavable in a detection assay, such as an invasive cleavage assay, by a flap endonuclease. In preferred embodiments, a flap oligonucleotide forms an invasive cleavage structure with other nucleic acids, e.g., a target or template nucleic acid and an invasive oligonucleotide. Flap assay reagents may optionally contain a target or template nucleic acid to which an invasive oligonucleotide and flap oligonucleotide bind. In particularly preferred embodiments, flap assay reagents comprise a Mg⁺⁺ flap assay buffer, as discussed herein.

As used herein, the term “flap endonuclease” refers to a structure-specific nucleolytic enzyme that cleaves a nucleic acid flap structure, e.g., an invasive cleavage structure. Flap endonuclease include, e.g., 5′ -exonuclease domains of the DNA polymerase I proteins of Eubacteria and the FEN-1 proteins of Eukarya and Archaea. (Kaiser, et al., supra). Flap endonucleases may cleave additional structures, e.g., pseudo-Y, 5′ overhang, and gap structures. See, e.g., Shen, B., BioEssays 27:717-729 (2005); Finger, L D., Subcell Biochem. 62:301-326 (2012), and U.S. Patent Appl. Ser. No. 62/901,085, filed Sep. 16, 2019, each of which is incorporated herein in its entirety. As used herein the term “flap endonuclease substrate” refers to a nucleic acid flap structure, e.g., an invasive cleavage structure, that is recognized and cleaved by a flap endonuclease, such as a FEN-1 endonuclease.

The term “FEN-1” as used herein in reference to an enzyme refers to a non-polymerase flap endonuclease from a eukaryote or archaeal organism, as encoded by a FEN-1 gene. See, e.g., Kaiser, et al., supra, WO 02/070755, and U.S. Pat. No. 7,122,364, which are incorporated by reference herein in their entireties for all purposes. The term “FEN-1 activity” refers to any enzymatic activity of a FEN-1 enzyme. FEN-1 endonucleases also comprise modified FEN-1 proteins, e.g., chimerical proteins comprising portions of FEN-1 enzymes from different organisms, and enzymes comprising one or more mutations (e.g., substitutions, deletions, insertions, etc.), as described in WO 02/070755, and U.S. Pat. No. 7,122,364.

As used herein, the terms “flap endonuclease assay” “flap assay” refer to a detection assay in which formation and cleavage of a flap endonuclease substrate is used to evaluate a sample for the presence of or an amount of a target analyte, e.g., a target nucleic acid.

As used herein, the term “flap assay reagents” or “invasive cleavage assay reagents” refers to a collection of all reagents required for performing a flap assay or invasive cleavage assay. As is known in the art, flap assays generally include oligonucleotides for forming an invasive cleavage structure, a flap endonuclease and, optionally, a FRET cassette or 5′ hairpin FRET reporter. Flap assay reagents may optionally contain a target to which the invasive oligonucleotide and flap oligonucleotide bind.

As used herein, the term “FRET cassette” refers to a hairpin oligonucleotide that contains a fluorophore moiety and a nearby quencher moiety that quenches the fluorophore. Hybridization of a cleaved flap (e.g., from cleavage of a target-specific probe in a PCR-flap assay assay) with a FRET cassette produces a secondary substrate for the flap endonuclease, e.g., a FEN-1 enzyme. Once this substrate is formed, the 5′ fluorophore-containing base can be cleaved from the cassette by the flap endonuclease, thereby generating a fluorescence signal. In preferred embodiments, a FRET cassette comprises an unpaired 3′ portion to which a cleavage product, e.g., a portion of a cleaved flap oligonucleotide, can hybridize to from an invasive cleavage structure cleavable by a FEN-1 endonuclease.

As used herein, the term “PCR-flap assay” is used interchangeably with the term “PCR-invasive cleavage assay” and refers to an assay configuration combining PCR target amplification and detection of the amplified DNA by formation of a first overlap cleavage structure comprising amplified target DNA, and a second overlap cleavage structure comprising a cleaved 5′ flap from the first overlap cleavage structure and a labeled reporter oligonucleotide, e.g., a “FRET cassette” or 5′ hairpin FRET reporter oligonucleotide. In the PCR-flap assay as used herein, the assay reagents comprise a mixture containing DNA polymerase, FEN-1 endonuclease, a primary probe comprising a portion complementary to a target nucleic acid, and a FRET cassette or 5′ hairpin FRET reporter, and the target nucleic acid is amplified by PCR and the amplified nucleic acid is detected simultaneously (i.e., detection occurs during the course of target amplification). PCR-flap assays include the QuARTS assays described in U.S. Pat. Nos. 8,361,720; 8,715,937; and 8,916,344, and the amplification assays of US Pat. No. 9,096,893 (for example, as diagrammed in FIG. 1 of that patent), each of which is incorporated herein by reference in its entirety.

As used herein, the term “PCR-flap assay reagents” refers to one or more reagents for detecting a target nucleic acid in a PCR-flap assay, the reagents comprising nucleic acid molecules capable of participating in amplification of a target nucleic acid and in formation of a flap endonuclease substrate in the presence of the target nucleic acid, preferably in a mixture containing DNA polymerase, FEN-1 endonuclease and a FRET cassette or 5′ hairpin FRET reporter.

As used herein, the term “FRET” refers to fluorescence resonance energy transfer, a process in which moieties (e.g., fluorophores) transfer energy e.g., among themselves, or, from a fluorophore to a non-fluorophore (e.g., a quencher molecule). In some circumstances, FRET involves an excited donor fluorophore transferring energy to a lower-energy acceptor fluorophore via a short-range (e.g., about 10 nm or less) dipole-dipole interaction. In other circumstances, FRET involves a loss of fluorescence energy from a donor and an increase in fluorescence in an acceptor fluorophore. In still other forms of FRET, energy can be exchanged from an excited donor fluorophore to a non-fluorescing molecule (e.g., a quenching molecule). FRET is known to those of skill in the art and has been described (See, e.g., Stryer et al., 1978, Ann. Rev. Biochem., 47:819; Selvin, 1995, Methods Enzymol., 246:300; Orpana, 2004 Biomol Eng 21, 45-50; Olivier, 2005 Mutant Res 573, 103-110, each of which is incorporated herein by reference in its entirety).

As used herein, the term “kit” refers to any delivery system for delivering materials. In the context of cell sampling devices, such delivery systems include systems that allow for the storage, transport, delivery, or use of devices and/or for processing samples obtained with devices (e.g., drinkable solutions, lubricants, or anesthetics for use of a swallowable device, sample stabilizing reagents; sample processing reagents such as particles, buffers, denaturants, oligonucleotides, filters, assay reaction components, etc. in the appropriate containers) and/or supporting materials (e.g., sample processing or sample storage vessels, written instructions for performing a procedure, etc.) from one location to another. For example, kits include one or more enclosures (e.g., boxes) containing the relevant sampling device and reagents and/or supporting materials. As used herein, the term “fragmented kit” refers to a delivery system comprising two or more separate containers that each contains a subportion of the total kit components. The containers may be delivered to the intended recipient together or separately. For example, a first container may contain materials for sample collection and a buffer. For instance, a first container may contain materials for sample collection and a cell stabilization buffer. A second container may contain reagents for detection of the one or more biomarkers. For example, a second container may contain capture oligonucleotides and denaturant. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” For example, a fragmented kit may contain analyte specific reagents, reagents for DNA extraction, and/or bisulfite conversion reagents. Alternatively, a fragmented kit comprising analyte specific reagents may be used in conjunction with a commercially available kit for DNA extraction. In such embodiments, the kit may comprise reagents for sample collection and a cell stabilization buffer, and may be used in conjunction with a suitable kit for DNA extraction to isolate DNA From the sample prior to detecting one or more biomarkers, such as biomarkers described herein. In contrast, a “combined kit” refers to a delivery system containing all of the components for sample collection, processing, and assaying in a single container (e.g., in a single box housing each of the desired components). The term “kit” includes both fragmented and combined kits.

The term “system” as used herein refers to a collection of articles for use for a particular purpose, e.g., a collection of devices, reagents, and instruments for collecting a sample (e.g., in preparation for analyzing the sample), or for collecting, processing and/or analyzing a sample for a particular purpose. In some embodiments, the articles of a system comprise instructions for use, as information supplied on e.g., an article, on paper, on recordable media (e.g., DVD, flash drive, etc.). In some embodiments, instructions direct a user to an online location, e.g., a website for viewing, hearing, and/or downloading instructions. In some embodiments, instructions or other information are provided as an application (“app”) for a mobile device.

### DESCRIPTION OF THE DRAWINGS

These and other features, aspects, and advantages of the present technology will become better understood with regard to the following drawings:

FIGS. 1A-1D show various embodiments of the dissolvable capsule described herein. FIG. 1A shows a capsule having a first closed end and a second closed end. FIG. 1B shows a capsule having a first closed end and a second open end. FIG. 1C shows a capsule having a first closed end and a second closed end and multiple openings. FIG. 1D shows a capsule having a first closed end and a second open end and multiple openings.

FIG. 2 shows an embodiment of the ingestible cell sampling device described herein. The device comprises an abrasive sponge housed in a compressed state within a dissolvable capsule having a first closed end and a second closed end, a spherical molded cap having an interior surface in contact with the exterior surface of the second closed end, and a suture attached to the molded cap.

FIG. 3 shows an embodiment of the ingestible cell sampling device described herein. The device comprises an abrasive sponge housed in a compressed state within a dissolvable capsule having a first closed end and a second closed end, a spherical molded cap having an interior surface in contact with the exterior surface of the second closed end, and a suture attached to the molded cap. The device further includes multiple openings along the cylindrical edge of the dissolvable capsule, such that the abrasive sponge is exposed to the external environment at these openings.

FIG. 4 shows an embodiment of the ingestible cell sampling device described herein. The device comprises an abrasive sponge housed in a compressed state within a dissolvable capsule having a first closed end and a second open end, a spherical molded cap having an interior surface in contact with the abrasive sponge, and a suture attached to the molded cap. The molded cap covers the second open end of the dissolvable capsule.

FIG. 5 shows an embodiment of the ingestible cell sampling device described herein. The device comprises an abrasive sponge housed in a compressed state within a dissolvable capsule having a first closed end and a second open end, a spherical molded cap having an interior surface in contact with the abrasive sponge, and a suture attached to the molded cap. The molded cap covers the second open end of the dissolvable capsule. The device further comprises multiple openings along the cylindrical edge of the dissolvable capsule.

FIGS. 6A-6D show various embodiments of the abrasive sponge described herein. FIG. 6A shows a cylindrical shaped abrasive sponge comprising a portion of material from the center removed. Approximately 25% of the material from the center of the sponge is removed. FIG. 6B shows a similar sponge wherein a larger portion of material from the center is removed. Approximately 50% of the material from the center of the sponge is removed. FIG. 6C shows a cylindrical sponge wherein multiple portions from the edge of the sponge are removed to create a pinwheel shape from the top view. FIG. 6D shows a cylindrical sponge wherein multiple portions from the edge of the sponge are removed to create a cross shape from the top view.

FIGS. 7A-7B show various views of an abrasive sponge wherein a portion (about 25%) of material from the center of the sponge is removed. The suture material passes through the abrasive sponge and attaches to the molded cap. The uncompressed diameter of the abrasive sponge is about 30 mm (FIG. 7A). A portion of material is removed from the center of the abrasive sponge, and the abrasive sponge is attached to the interior surface of the molded cap by an adhesive (FIG. 7B).

FIGS. 8A-8B show various views of an abrasive sponge wherein a portion (about 50%) of material from the center of the sponge is removed. The suture material passes through the abrasive sponge and attaches to the molded cap. The uncompressed diameter of the abrasive sponge is about 30 mm (FIG. 8A). A portion of material is removed from the center of the abrasive sponge, and the abrasive sponge is attached to the interior surface of the molded cap by an adhesive (FIG. 8B).

FIGS. 9A-9B show various views of an abrasive sponge wherein multiple portions of material from the edge of the sponge are removed. The uncompressed diameter of the abrasive sponge is about 30 mm (FIG. 9A). Multiple portions of material from the edge of the sponge are removed, to generate a sponge having a pinwheel shape from the top view (FIG. 9B).

FIGS. 10A-10B show various views of an abrasive sponge wherein multiple portions of material from the edge of the sponge are removed. The uncompressed diameter of the abrasive sponge is about 30 mm (FIG. 10A). Multiple portions of material from the edge of the sponge are removed, to generate a sponge having a cross shape from the top view (FIG. 10B).

FIGS. 11A-11D show various embodiments of methods for attaching the string to the molded cap. FIG. 11A shows an embodiment where the suture is attached to the molded cap by means of a knot. The molded cap sits on the outside of a closed end of the capsule. FIG. 11B shows an embodiment where the molded cap covers an open end of the capsule. The string is attached to the molded cap by means of a knot, and the molded cap comprises an elongated cylindrical edge, the circumference of which fits within the circumference of the open end of the capsule. FIG. 11C shows an embodiment where the molded cap is a button. The button fits within the capsule and the string is attached to the button by means of a knot. FIG. 11D shows an embodiment where the molded cap is a button. The button fits within the capsule and the string is attached to the button by means of a knot.

FIG. 12 shows multiple views of the embodiment for attachment shown in FIG. 11A. The molded cap comprises two holes through which the string is threaded (left). The knot to secure the string to the cap is tied on the inside of the molded cap (center). The molded cap fits over a closed end of the dissolvable capsule, such that the interior surface of the molded cap is in contact with the exterior surface of the closed end of the capsule (right).

FIG. 13 shows multiple views of the embodiment for attachment shown in FIG. 11B. The molded cap comprises two holes through which the string is threaded (left). The knot to secure the string to the cap is tied on the inside of the molded cap (center). The molded cap comprises an elongated cylindrical edge, the circumference of which fits within the circumference of the open end of the capsule (center, right). The exterior surface of the molded cap is in contact with the external environment.

FIG. 14 shows multiple views of the embodiment for attachment shown in FIG. 11C. The molded cap is a button. The button comprises two holes through which the string is threaded (left). The knot to secure the string to the button is tied on the inside of the molded cap (center). Alternatively or in addition, the string may be secured to the molded cap by use of an adhesive. The button fits within the capsule (right).

FIG. 15 shows multiple views of the embodiment for attachment shown in FIG. 11D. The molded cap is a button. The button comprises a bar feature molded into the cap (left). The string wraps around the bar feature, and the knot to secure the string to the button is tied on the inside of the molded cap (center). Alternatively or in addition, the string may be secured to the button (e.g., to the bar feature) by an adhesive. The button fits within the capsule (right).

FIG. 16 shows multiple views of an exemplary embodiment of the ingestible cell sampling device as described herein. The device comprises dissolvable capsule having a first closed end and a second closed end. The device comprises a spherical molded cap having an exterior surface in contact with the interior surface of the second closed end, and a suture attached to the molded cap. An abrasive sponge may be housed in a compressed state within the dissolvable capsule, such that the interior surface of the spherical molded cap is in contact with the abrasive sponge. Exemplary side, top, and angled views of the molded cap are shown on the right. The molded cap comprises two holes to allow attachment of the suture, and is slightly recessed on top to account for suture thickness.

FIG. 17 shows multiple views of an exemplary embodiment of the ingestible cell sampling device as described herein. The device comprises dissolvable capsule having a first closed end and a second closed end. The device comprises a spherical molded cap having an exterior surface in contact with the interior surface of the second closed end, and a suture attached to the molded cap. An abrasive sponge may be housed in a compressed state within the dissolvable capsule, such that the interior surface of the spherical molded cap is in contact with the abrasive sponge. Exemplary side, top, and angled views of the molded cap are shown on the right. The molded cap comprises two holes to allow attachment of the suture, and is slightly recessed on top to account for suture thickness. The holes are slightly larger than the holes shown in the embodiment of FIG. 16. The larger holes for this device compared to those shown in FIG. 16 may permit different (e.g. larger) knots to be used to attach the suture to the molded cap.

FIG. 18 shows multiple views of an exemplary embodiment of the ingestible cell sampling device as described herein. The device comprises dissolvable capsule having a first closed end and a second open end. An abrasive sponge may be housed in a compressed state within the dissolvable capsule. The device comprises a spherical molded cap having an interior surface in contact with the abrasive sponge, and a suture attached to the molded cap. The exterior surface of the spherical molded cap is exposed to the external environment. The molded cap covers the second open end of the dissolvable capsule. Exemplary side, top, and angled views of the molded cap are shown on the right. The molded cap comprises two holes to allow attachment of the suture, and is slightly recessed on top to account for suture thickness.

FIG. 19 shows multiple views of an exemplary embodiment of the ingestible cell sampling device as described herein. The device comprises dissolvable capsule having a first closed end and a second open end. An abrasive sponge may be housed in a compressed state within the dissolvable capsule. The device comprises a spherical molded cap having an interior surface in contact with the abrasive sponge, and a suture attached to the molded cap. The exterior surface of the molded cap is exposed to the external environment. The molded cap covers the second open end of the dissolvable capsule. Exemplary side, top, and angled views of the molded cap are shown on the right. The molded cap comprises two holes to allow attachment of the suture, and is slightly recessed on top to account for suture thickness. The holes are slightly larger than the holes shown in the embodiment of FIG. 18. The larger holes for this device compared to those shown in FIG. 18 may permit different (e.g. larger) knots to be used to attach the suture to the molded cap. FIG. 20 shows multiple views for an exemplary embodiment of a handle as described herein. The handle has a hook shape. The handle “pinches” the dissolvable capsule within a set of grippers at one end of the handle. The other end of the handle is a hook. The suture may be wound around any suitable portion of the handle. For use in a subject, the ingestible device may be removed and the suture may be unwound. The device may be ingested by the subject, while the subject or a third party holds on to the hook end of the handle.

FIG. 21 shows an exemplary embodiment of a handle as described herein. The handle comprises a cavity in which the dissolvable capsule may be placed. The suture may be wound around a separate portion of the handle, as shown. When wound, the suture may be held in place by a suitable amount of tension. The handle may comprise tabs, which may be squeezed to remove tension from the suture and allow for facile removal of the suture from the handle, without the need to unwind the entire length of the suture.

FIG. 22 shows another exemplary embodiment of a handle as described herein. The handle may be circular in shape. The handle comprises a cavity in which the dissolvable capsule may be placed. The suture may be wound around the external edge of the circular handle, such as along a slightly recessed channel extending along the outer edge of the handle. The suture may be tied in such as a position, which may be facilitated by a single hole placed in the circular handle. The suture may be untied and unwound from the circular handle to allow for the subject to ingest the device.

FIG. 23 shows another exemplary embodiment of a handle as described herein. The handle is a T-shape. The handle comprises a cavity in which the dissolvable capsule may be placed. The suture may be held in place by winding the suture around the handle.

FIG. 24 shows an exemplary embodiment of a handle as described herein. The handle comprises a flat surface on one end, and a hook shape on the opposing end. The flat surface comprises a cavity in which the dissolvable capsule may be placed. The flat surface additionally comprises a plurality of openings to provide a variety of suitable attachment sites for the suture.

FIGS. 25A-25F show exemplary assay designs for detecting biomarkers of esophageal disorders in a sample.

### DETAILED DESCRIPTION OF THE INVENTION

The technology relates to cell sampling devices. In particular, the present invention relates to ingestible cell sampling devices and their use in methods for detecting various abnormalities in a subject.

The ingestible cell sampling devices described herein are advantageous in that the devices provide improved safety for use in a subject. For example, the ingestible cell sample devices described herein comprise a handle to facilitate ease of use by the subject. The handle provides a suitable surface for the user or a third party to hold onto during ingestion of the dissolvable capsule component of the device, thereby preventing loss within the subject and facilitating facile removal of the device following a suitable duration of time. As another example, the ingestible cell sampling devices described herein are designed to prevent detachment of the string from the molded cap, thus preventing loss of the device within a subject. As another example, the ingestible cell sampling devices described herein are designed to minimize the risk of the sponge detaching from the string during retrieval of the device, thus also preventing loss of the device within the subject. Furthermore, the devices described herein use materials that prevent laceration to the esophagus upon withdrawal of the device. The devices are easily swallowed by the subject, with a rapid dissolution of the capsule and expansion of the sponge, thus minimizing the total time required to collect an esophageal sample from the subject. Furthermore, the sponge comprises multiple features that enable for maximal surface area to capture sufficient tissue from a subject. Accordingly, described herein are ingestible cell sampling devices with maximal sampling capabilities having enhanced safety and tolerability for use in a subject.

In some embodiments, provided herein are ingestible cell sampling devices. The devices comprise an abrasive sponge housed within a dissolvable capsule, a molded cap, and a string attached to the molded cap.

The abrasive sponge may comprise any suitable material. Preferably, the material is capable of being compressed and retained in a compressed state by the dissolvable capsule. For example, the abrasive sponge may comprise a reticulated material. In some embodiments, the reticulated material comprises 10-35 pores per inch of material. For example, the reticulated material may comprise about 10, about 15, about 20, about 25, about 30, or about 35 pores per inch of material. The material may be any suitable porous, low-density material capable of collecting esophageal cells from a subject. For example, the abrasive sponge may comprise reticulated polyurethane. In some embodiments, the abrasive sponge comprises a reticulated polyester material. In some embodiments, the abrasive sponge comprises a reticulated polyether material.

The porosity of the abrasive sponge may be at least 80%. For example, the porosity may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

The abrasive sponge may be of any suitable size and shape. The size and shape of the sponge may depend on the size of the capsule. In some embodiments, the abrasive sponge is of a suitable size and shape to allow for compression into a capsule suitable for oral administration (e.g. ingestion), and subsequent facile removal from the esophagus and throat of a subject after dissolution of the capsule and restoration of the sponge to its uncompressed size. For example, the sponge may be cylindrical in shape. For example, the sponge may be cylindrical in shape with a diameter (e.g. the diameter of the circular portion forming the shaft of the cylinder) of about 20-400 mm in an uncompressed shape. For example, the diameter may be about 20 mm, about 25 mm, about 30 mm, about 35 mm, or about 40 mm in an uncompressed state. For example, the sponge may be cylindrical in shape with a diameter of 20 mm, 21 mm, 22 mm, 23 mm, 24 mm, 25 mm, 26 mm, 27 mm, 28 mm, 29 mm, or 30 mm in an uncompressed state.

As another example, the sponge may be spherical in shape. For example, the sponge may be spherical in shape with a diameter of about 20-40 mm in an uncompressed state. For example, the sponge may be spherical in shape with a diameter of about 20 mm, about 25 mm, about 30 mm, about 35 mm, or about 40 mm in an uncompressed state. For example, the sponge may be spherical in shape with a diameter of 20 mm, 21 mm, 22 mm, 23 mm, 24 mm, 25 mm, 26 mm, 27 mm, 28 mm, 29 mm, or 30 mm in an uncompressed state.

The abrasive sponge may be compressed to a suitable size and housed within the dissolvable capsule in a compressed state. For example, the compressed sponge may have a diameter of about 1 mm to about 15 mm. For example, the compressed sponge may have a diameter of 1 mm, 2 mm, 3 mm, 4 mm, 5 mm, 6 mm, 7 mm, 8 mm, 9 mm, 10 mm, 11 mm, 12 mm, 13 mm, 14 mm, or 15 mm in a compressed state.

In some embodiments, the dissolution of the dissolvable capsule releases the abrasive sponge from compression and allows the abrasive sponge to expand. In some embodiments, the abrasive sponge expands to its uncompressed size following dissolution of the dissolvable capsule. In some embodiments, the abrasive sponge expands to substantially the same size as its original, uncompressed size prior to packaging within the capsule. As a nonlimiting example, the abrasive sponge may expand to within 10% of the original, uncompressed size following dissolution of the dissolvable capsule. For example, the sponge may expand to within 1%, 2%, 3%, 4%, 5%, 6%, 7%, 8%, 9%, or 10% of the original, uncompressed size following dissolution of the dissolvable capsule. For example, the original, uncompressed size may be 30 mm and the sponge may expand to 27-30 mm following dissolution of the capsule.

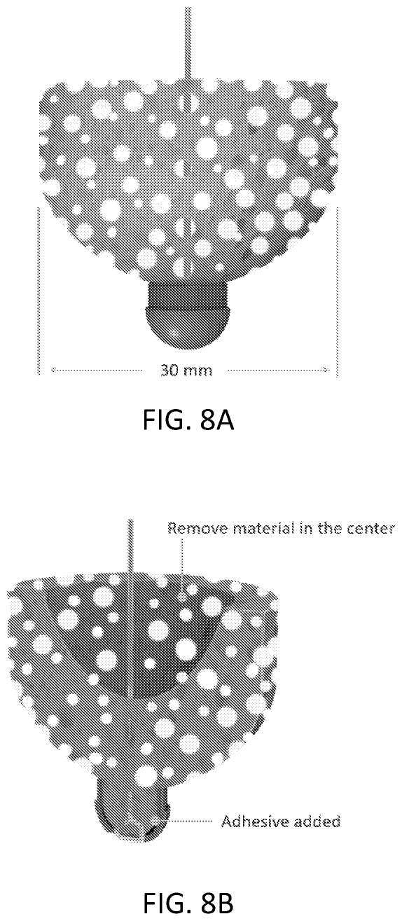

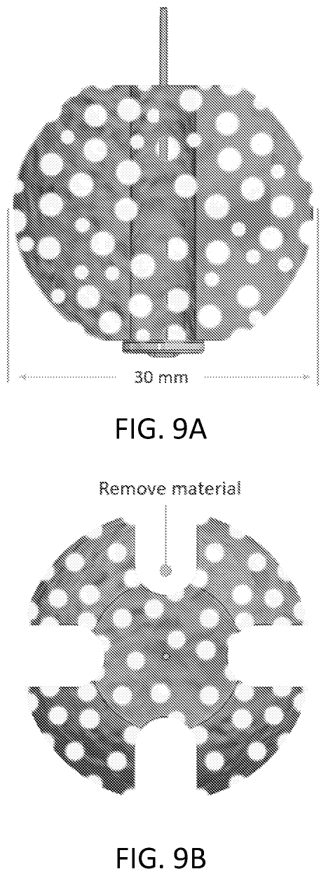

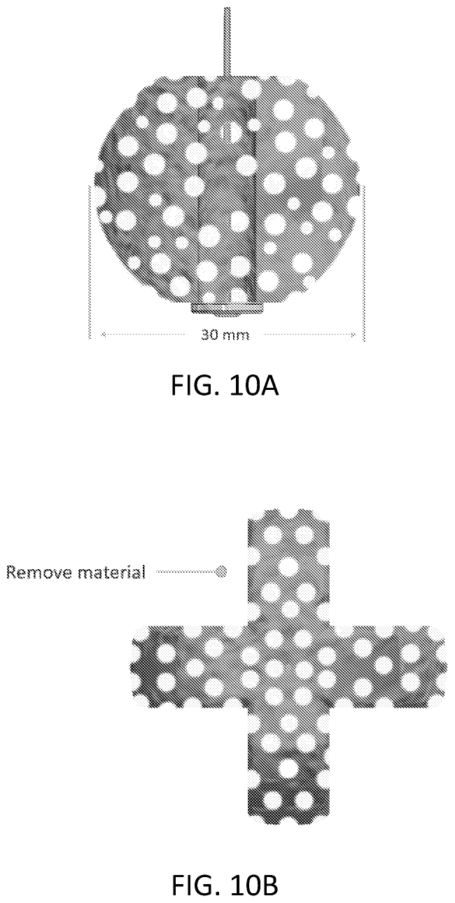

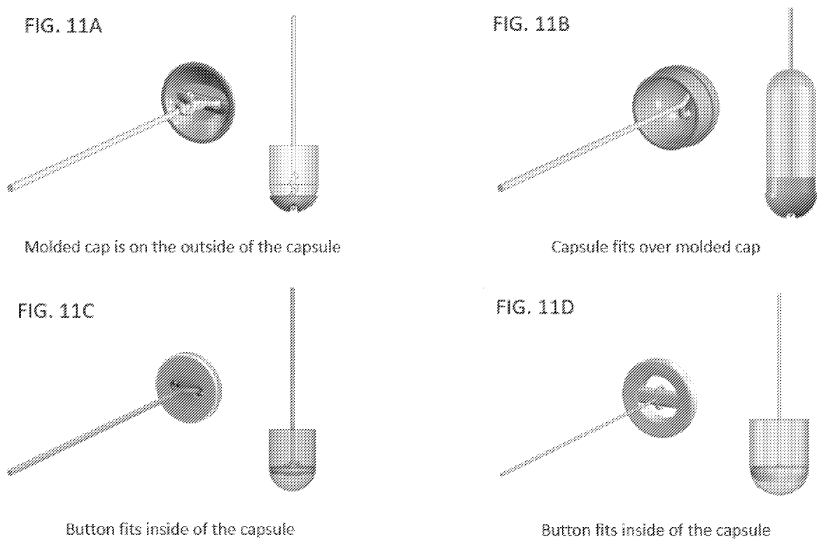

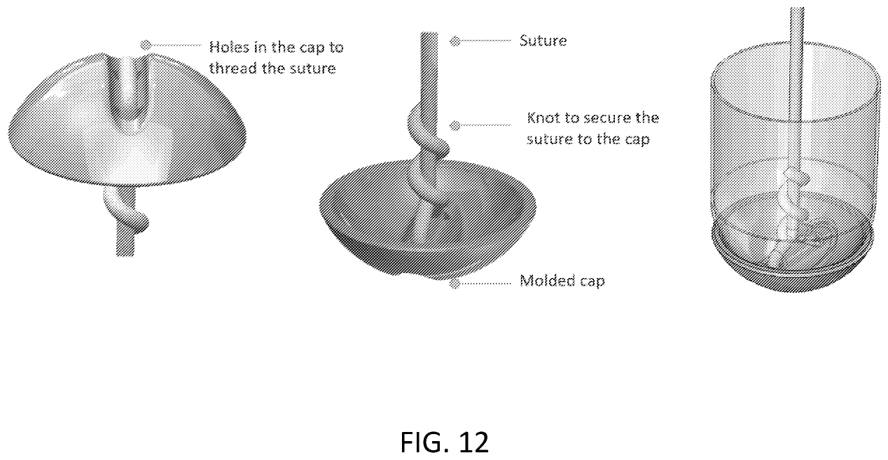

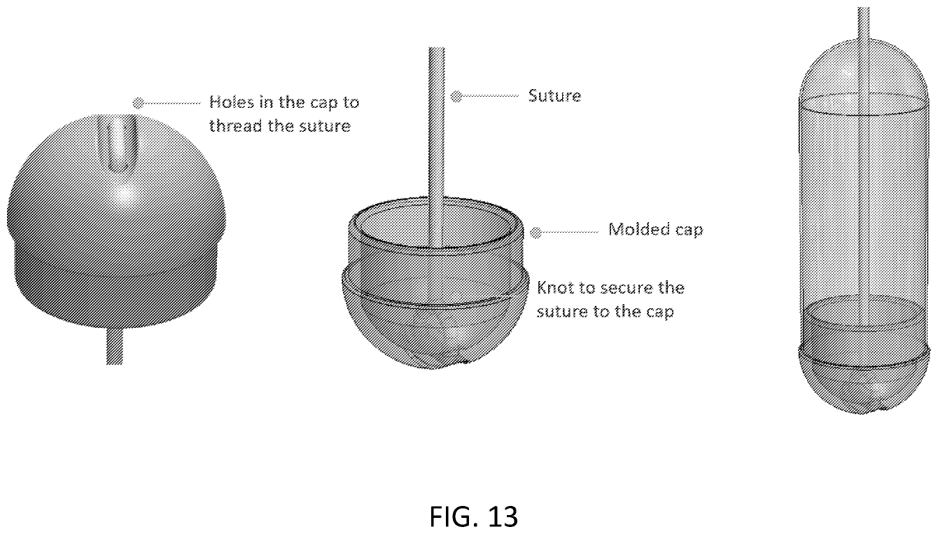

In some embodiments, the uncompressed sponge is uniform in shape. For example, the uncompressed sponge may be uniformly spherical in shape. In some embodiments, the uncompressed sponge may be uniformly cylindrical in shape. In some embodiments, the uncompressed sponge may be spherical in shape with a protrusion that extends a portion of the abrasive sponge material into the dissolvable capsule. This protrusion is exemplified in FIG. 8B.

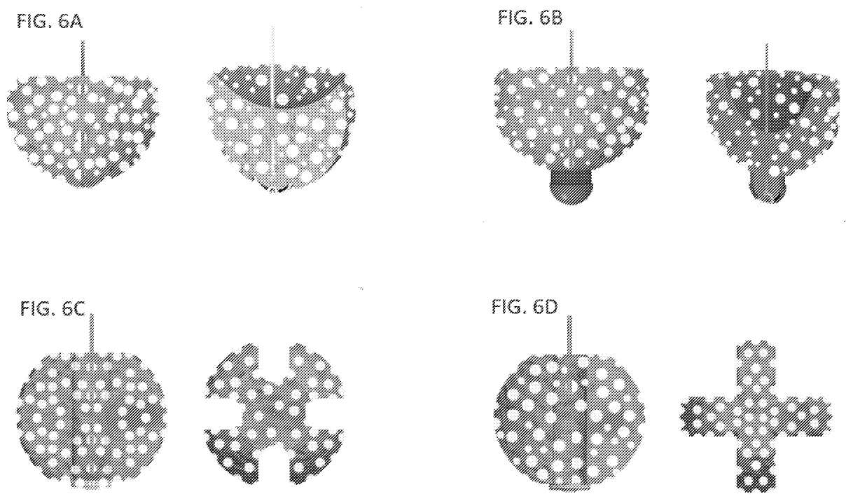

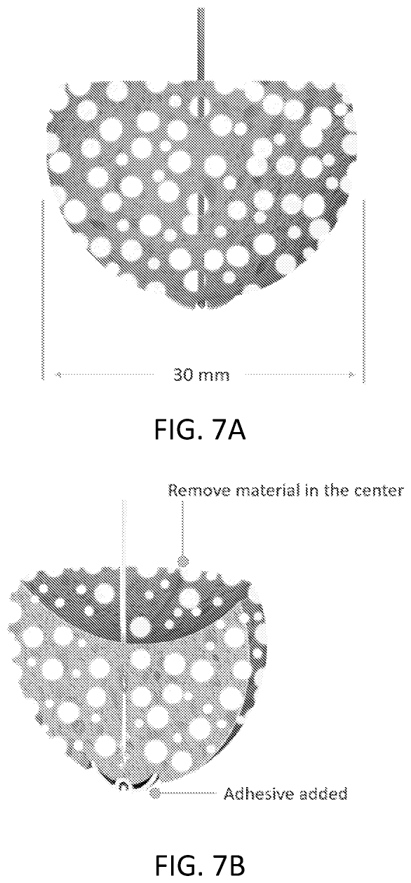

In some embodiments the abrasive sponge is formed to have concavities or indentations, or other external or internal spaces devoid of sponge material (“void spaces”), as may be provided by removing at least one portion of the abrasive sponge. As used herein in reference to a shape of an abrasive sponge, a “removed” portion of a sponge refers a void space in the shaped abrasive sponge, e.g., a portion that would be removed from a simple solid form, e.g., a sphere or cylinder, to produce the final shape comprising one or more void spaces. It is understood that an abrasive sponge may be manufactured in a final form that comprises such a void space, such that no sponge material need be physically “removed” during manufacture. In some embodiments, at least one portion of material may be removed from the center of the abrasive sponge. For example, the sponge may be cylindrical in shape and a portion of the material may be removed from the center of the sponge. As another example, the sponge may be spherical in shape and a portion of the material may be removed from the center of the abrasive sponge. For example, such embodiments are exemplified in FIGS. 6A and 6B. In some embodiments, at least one portion of material may be removed from at least one outer edge of the abrasive sponge. For example, the sponge may be cylindrical in shape and at least one portion of the material may be removed from the edge of the abrasive sponge. As another example, the sponge may be spherical in shape and at least one portion of the material may be removed from the edge of the abrasive sponge. Various embodiments are exemplified in FIGS. 6C and 6D. For example, multiple portions of material may be removed from the edge of the sponge to generate a pinwheel shape (as shown in FIG. 6C) or a cross-shape (as shown in FIG. 6D), when the sponge is viewed from the top.

Any suitable sized portion may be removed from the sponge. For example, 5%, 10%, 15%, 20%, 25%, 30%, 35%, 40%, 45%, 50%, or more of the material may be removed from the sponge. In some embodiments, removal of a portion of the material facilitates compression of the sponge into a suitable sized capsule for ingestion by a subject. In some embodiments, removal of a portion of the material from the sponge facilitates rapid expansion of the sponge following dissolution of the dissolvable capsule. In some embodiments, removal of a portion of the material from the sponge increases the surface area of the sponge available for collecting esophageal cells in the subject.

The abrasive sponge is housed within a dissolvable capsule. Accordingly, the abrasive sponge may be compressed into a cylindrical shape to fit into the dissolvable capsule. The dissolvable capsule may comprise any suitable material. For example, the dissolvable capsule may comprise gelatin, starch, or a cellulosic material as known in the art. In some embodiments, the dissolvable may be a vegan or vegetarian capsule (e.g., exclusive of all animal products, or free of products from certain types of animals; e.g. gelatin-free capsule).

The dissolvable capsule may be made of a suitable material. In some embodiments, the dissolvable capsule comprises a suitable material that dissolves within 10 minutes of entering the stomach cavity of a subject. For example, the dissolvable capsule may dissolve approximately within 10 minutes, within 9 minutes, within 8 minutes, within 7 minutes, within 6 minutes, within 5 minutes, within 4 minutes, within 3 minutes, within 2 minutes, or within 1 minute of exposure to the stomach cavity of a subject. Preferably, the dissolvable capsule dissolves within 5 minutes of exposure to the stomach cavity of the subject.

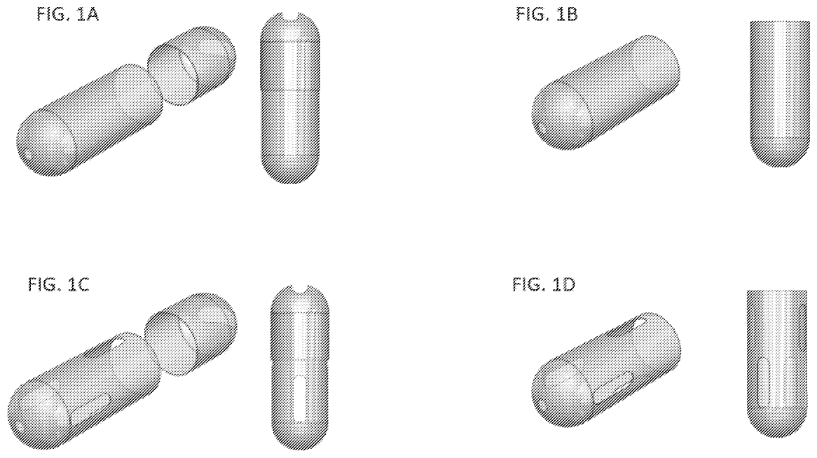

In some embodiments, the dissolvable capsule comprises a first closed end and a second closed end. For example, a dissolvable capsule comprising a first closed end and a second open end is shown in FIG. 1A. In other embodiments, the dissolvable capsule comprises a first closed end and a second open end. For example, a dissolvable capsule comprising a first closed end and a second open end is shown in FIG. 1B.

In some embodiments, the dissolvable capsule comprises one or more openings, such that a portion of the abrasive sponge is exposed to the external environment at the one or more openings. The presence of one or more openings may facilitate a faster dissolution time of the capsule upon ingestion by a subject. In some embodiments, the dissolvable capsule comprises one opening. In some embodiments, the dissolvable capsule comprises two or more openings. Representative images of capsules containing one or more openings are shown in FIG. 1C (capsule having a first closed end and a second open end) and FIG. 1D (capsule having a first closed end and a second open end).

The one or more openings may be any suitable size and shape that allow for exposure of the abrasive sponge to the external environment without substantially diminishing the ability of the capsule to retain the sponge in a compressed state. The one or more openings may be in any suitable location on the dissolvable capsule. For example, the dissolvable capsule may comprise one or more openings on a closed end of the capsule. As another example, the dissolvable capsule may comprise one or more openings on a cylindrical edge of the capsule.

The ingestible cell sampling device further comprises a molded cap. In some embodiments, the molded cap is hemispherical in shape. In some embodiments, the molded cap is cylindrical in shape (e.g., a button). In some embodiments, the molded cap is connected to the capsule. For example, the molded cap may be connected to the capsule by an adhesive. In some embodiments, the molded cap is connected to the abrasive sponge. For example, the molded cap may be connected to the abrasive sponge by an adhesive. In some embodiments, the molded cap fits within the capsule.

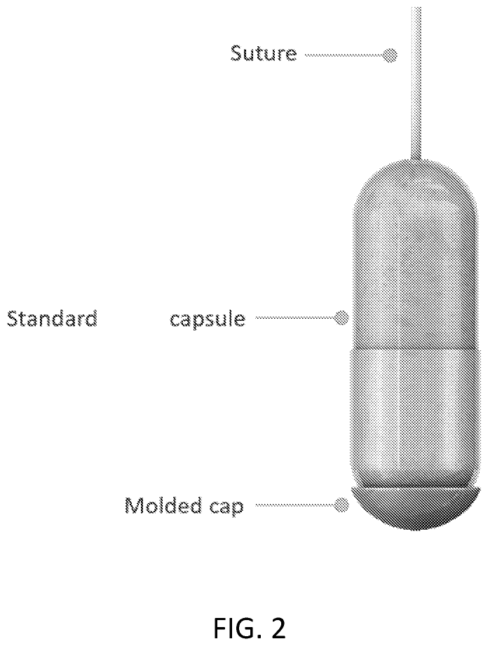

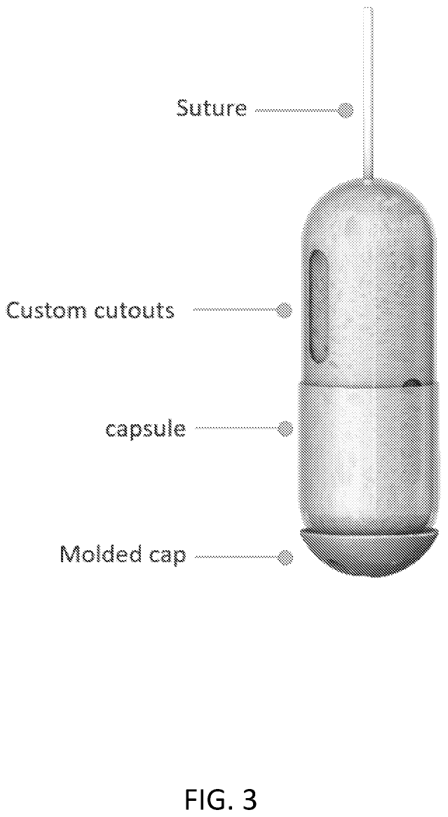

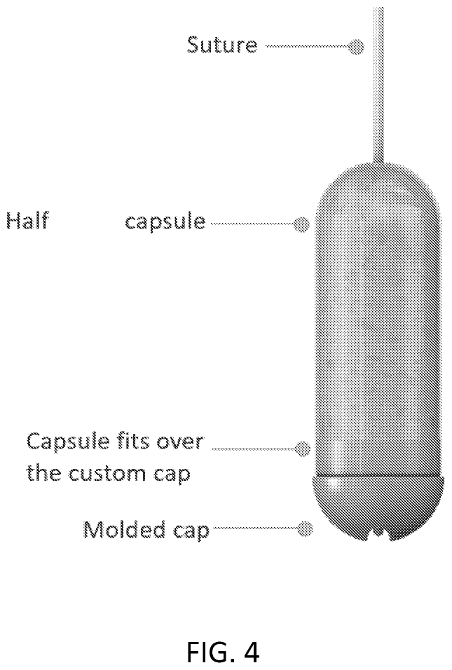

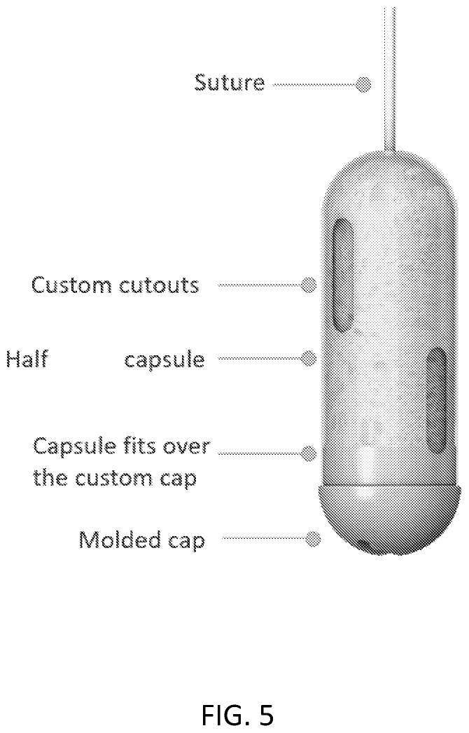

In some embodiments, the cell sampling device may comprise a capsule comprising a first closed end and a second closed end, and a hemispherical molded cap may cover one of the closed ends of the capsule. For example, the molded cap may comprise an internal surface in contact with an external surface of one end of the capsule and an exterior surface in contact with the external environment (as exemplified in FIG. 2 and FIG. 3). In some embodiments, the molded cap comprises an interior surface in contact with the abrasive sponge and an exterior surface. In some embodiments, the exterior surface may be in contact with the external environment. For example, the capsule may comprise a first closed end and a second open end, and the molded cap may cover the second open end of the capsule. For example, the molded cap may comprise an elongated cylindrical edge, the circumference of which fits within the circumference of the capsule (as exemplified in FIG. 4).

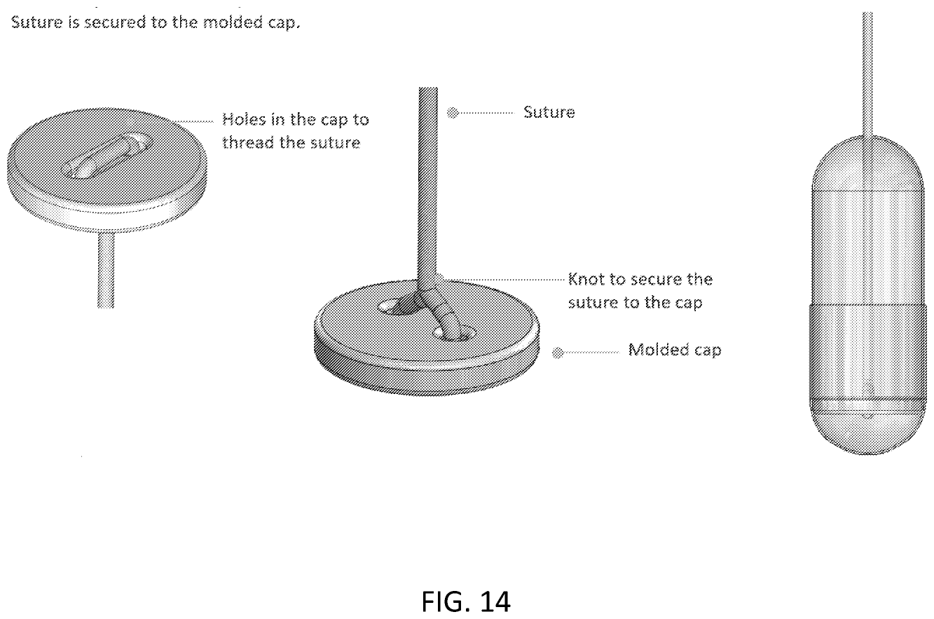

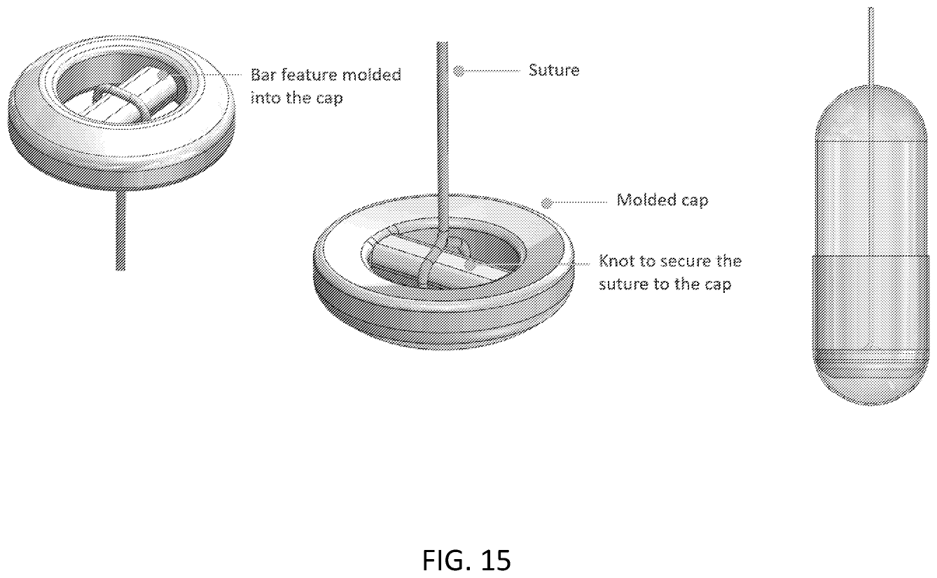

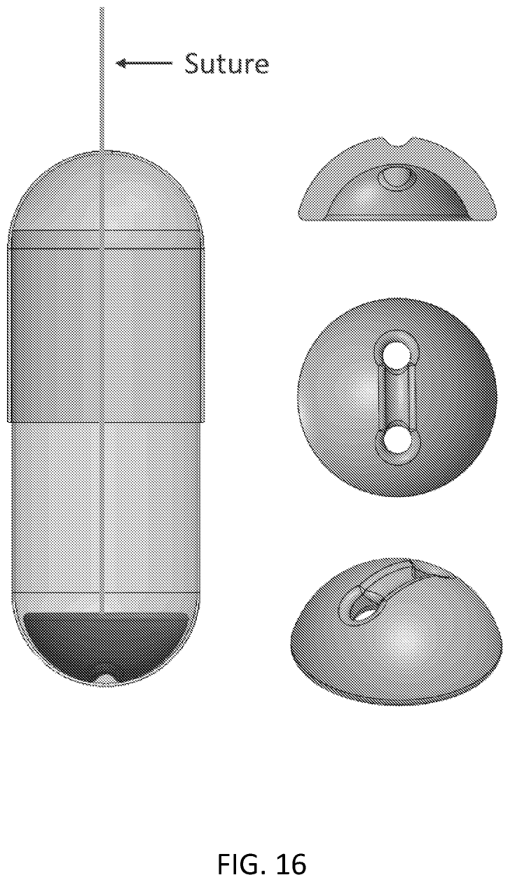

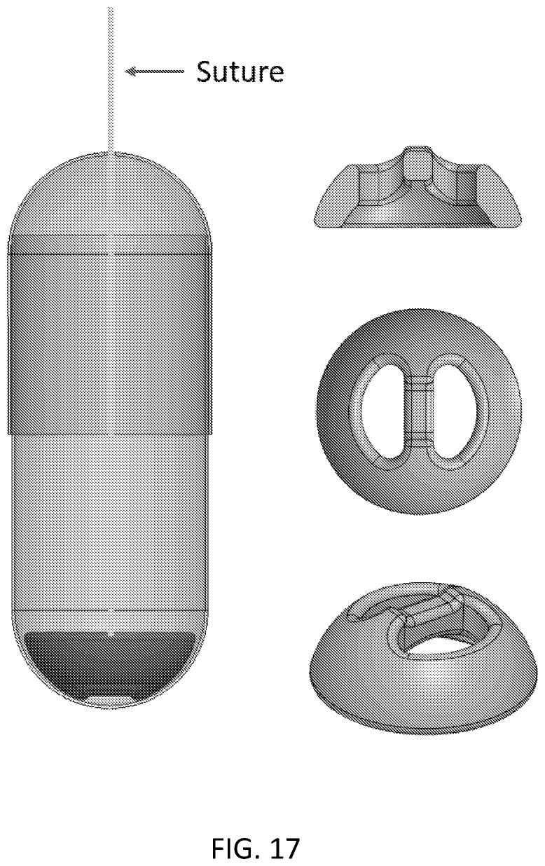

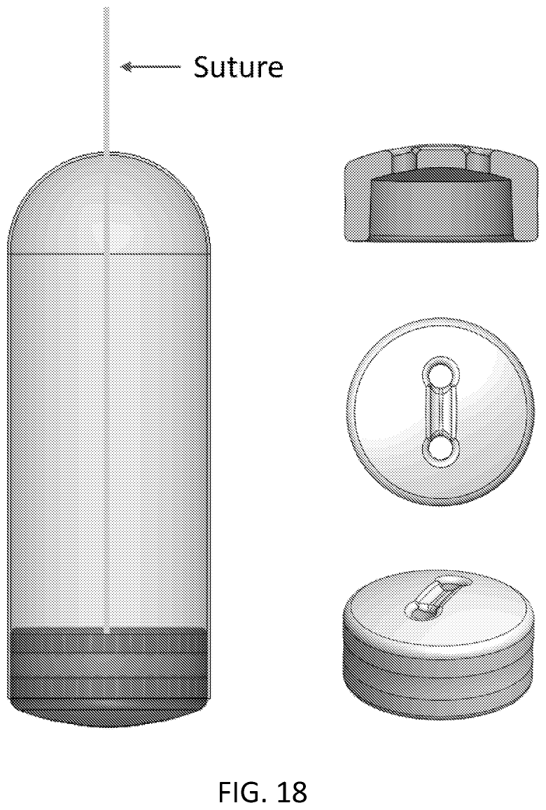

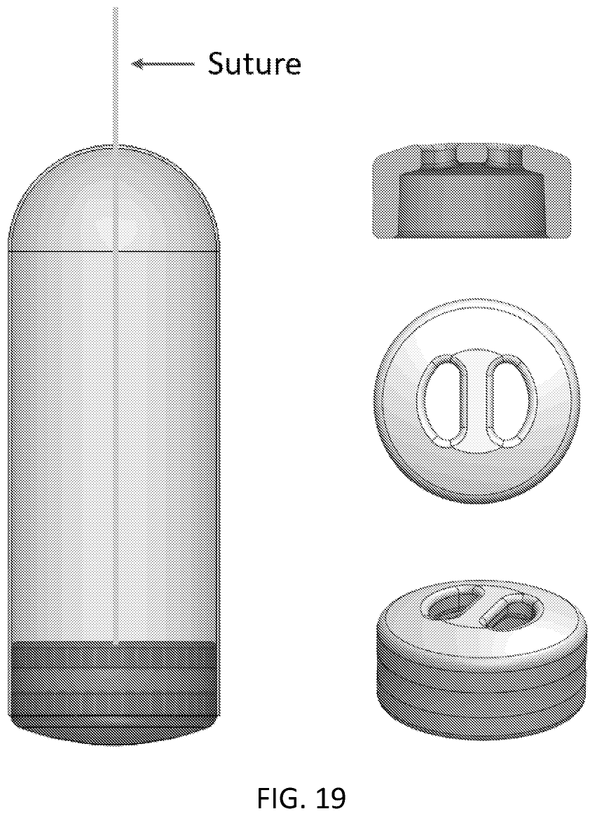

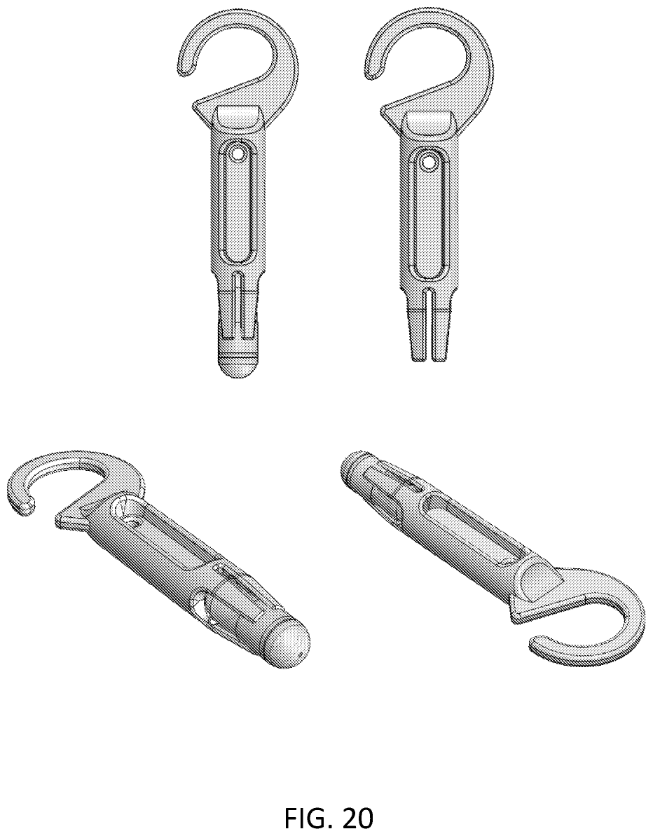

In some embodiments, the molded cap comprises an interior surface in contact with the abrasive sponge and an exterior surface in contact with an internal surface of one end of the capsule. For example, the capsule may comprise a first closed end and a second closed end, and a hemispherical molded cap may comprise an interior surface in contact with the abrasive sponge and an exterior surface in contact with the internal surface of one closed end of the capsule (e.g., the molded cap is fitted within the capsule). In some embodiments, the capsule may comprise a first closed end and a second closed end, and a cylindrically shaped molded cap (e.g., a button) may be fitted inside the capsule. In such embodiments, the button may be fitted inside the capsule such that the rim of the molded cap is in contact with the capsule, the bottom surface of the molded cap is in contact with the abrasive sponge, and the top surface of the molded cap is not in direct contact with the interior surface of the capsule (as exemplified in FIG. 14 and FIG. 15). In some embodiments, the cell sampling device comprises a capsule comprising a first closed end and a second closed end, and a hemispherical molded cap may fit within the capsule. For example, the cell sampling may comprise a hemispherical molded cap having an exterior surface in contact with the interior surface of the second closed end, and a suture attached to the molded cap. An abrasive sponge may be housed in a compressed state within the dissolvable capsule, such that the interior surface of the spherical molded cap is in contact with the abrasive sponge. Such an embodiment is shown, for example, in FIG. 16 and FIG. 17.

In embodiments wherein the interior surface of the molded cap is in contact with the abrasive sponge, the interior surface of the molded cap may be attached to the abrasive sponge. For example, the interior surface of the molded cap may be attached to the abrasive sponge by an adhesive.

In embodiments where a surface of the molded cap is in contact with the capsule, the molded cap may be attached to the capsule (e.g. by an adhesive).

The ingestible cell sampling device further comprises a string attached to the molded cap. The string may be attached to the molded cap by any suitable means, including but not limited to crimping, over-molding, adhesive, melting, wrapping, or taping. In some embodiments, the string is attached to the molded cap by an adhesive. In some embodiments, the string is attached to the molded cap by a knot. Any suitable type of knot may be used. For example, the knot may be a hitch knot. The term “hitch knot” refers to a type of knot used to tie a string to an object or to another string. The term encompasses many distinct types of hitch knots, including an alternate ring hitching, anchor bend variant, bale sling hitch, barrel hitch, becket hitch, blackwall hitch, blake's hitch, boom hitch, bottom loaded release hitch, buntline hitch, cat's paw, chain hitch, clinging clara, clove hitch, continuous ring hitching, cow hitch variant, cow hitch with toggle, cow hitch, double half hitches, Farrimond friction hitch, garda hitch, ground-line hitch, half hitch, halter hitch, highpoint hitch, highwayman's hitch, hitching tie, icicle hitch, killick hitch, knute hitch, lighterman's hitch, magnus hitch, marline hitching, marlinespike hitch, masthead knot, midshipman's hitch, munter hitch, munter friction hitch, ossel hitch, palomar knot, pile hitch, prusik knot, reverse half hitches, round hitch, round turn and two half hitches, sailor's gripping hitch, sailor's hitch, siberian hitch, single hitch, slippery hitch, snell knot, snuggle hitch, taut-line hitch, timber hitch, trilene knot, trucker's hitch, tugboat hitch, uni knot, or a wagoner's hitch knot. In some embodiments, the hitch knot is a double overhand knot.

In some embodiments, the knot may be a binding knot. The term “binding knot” refers to a type of knot used to keep an object or multiple objects together, using a string that passes at least once around them. Suitable binding knots include, for example, a boa knot, a bottle sling, a bowline knot, a constrictor knot, a corned beef knot, a granny knot, a ground-line hitch, a Miller's knot, a Packer's knot, a reef knot, a strangle knot, a surgeon's knot, a thief knot, a jamming knot, a sheet bend, or a common whipping knot. The type of knot may be selected to allow for ease of manufacturing while also providing a stable means of connecting the string to the molded cap.

The molded cap may comprise any suitable feature to enable attachment of the string to the cap. For example, the molded cap may comprise two holes through which the string can be threaded and tied into a suitable knot. The string can be threaded through the first hole, pass through the external environment, and re-enter the interior of the capsule by passing through the second hole, before a knot can be tied on the interior of the capsule. As another example, the molded cap may comprise a bar on which the string can be secured (as shown in FIG. 11D).

The string may comprise any suitable material. For example, the string may be a suture material (e.g. surgical suture material). The suture material may be made from a variety of materials, including biological materials or synthetic materials. For example, the suture material may comprise synthetic materials such as nylon, polyester, PVDF, polypropylene, or combinations thereof.

The string should be of a suitable thickness to allow for facile ingestion by the subject without causing lacerations to the throat. In some embodiments, the string has a thickness of 0.3 mm to 0.7 mm. For example, the string may have a thickness of 0.3 mm, 0.35 mm, 0.4 mm, 0.45 mm, 0.5 mm, 0.55 mm, 0.6 mm, 0.65 mm, or 0.7 mm.

The string should be of a suitable length to allow for retrieval of the device after dissolution of the dissolvable capsule in the subject. Accordingly, the string should be long enough to allow for the abrasive sponge contained within the dissolvable capsule to reach the stomach cavity of the subject, while retaining enough string for the subject or a physician to be able to grip the string to initiate retrieval of the device. For example, the string may be at least 60 cm long. In some embodiments, the string may be 60cm to 80cm long. For example, the string may be 60 cm, 61 cm, 62 cm, 63 cm, 64 cm, 65 cm, 66 cm, 67 cm, 68c cm, 69 cm, 70 cm, 71 cm, 72 cm, 73 cm, 74 cm, 75 cm, 76 cm, 77 cm, 78 cm, 79 cm, or 80 cm long.

In some embodiments, the string may comprise markings on the string to judge the amount of string that has been swallowed. Such markings would assist in determining that the dissolvable capsule has traveled to the desired area (e.g. the stomach cavity of the subject). The markings may be spaced any suitable distance apart. For example, the markings may be spaced 1-80 cm apart. For example, the markings may be placed about 1 cm, about 5 cm, about 10 cm, about 15 cm, about 20 cm, about 25 cm, about 30 cm, about 35 cm, or about 40 cm apart.

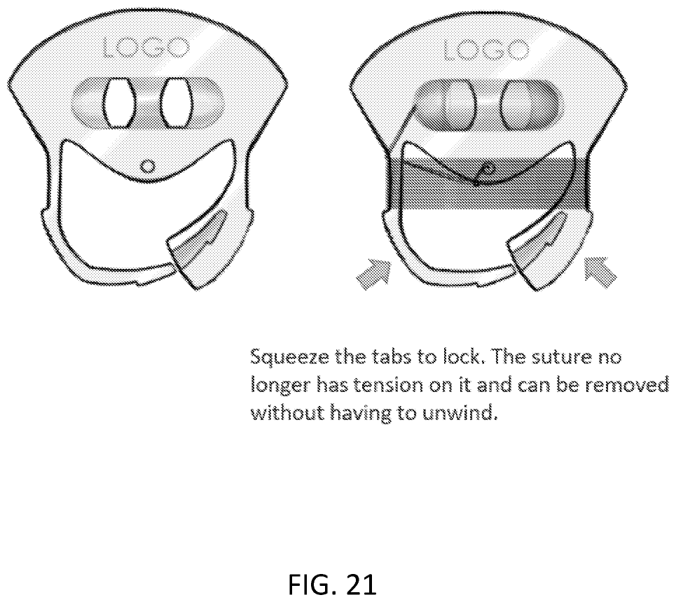

The string should have a suitable tensile strength to minimize the risk of the string breaking during ingestion and/or retrieval of the device. For example, the string should have a suitable tensile strength to allow for the string to be pulled to retrieve the device from the subject after dissolution of the dissolvable capsule. In some embodiments, the ingestible device may comprise a handle or a grip to facilitate retrieval of the device and/or prevent swallowing of the entire string. For example, the ingestible device may comprise a handle attached to the end of the string that is not attached to the moldable cap or button. For example, the ingestible device may comprise a handle or a grip on the end of the string that does not contain the capsule. The handle or grip may be of any suitable size and shape to facilitate retrieval and prevent swallowing of the string. The handle or grip may be an open shape (e.g. bar shape, T-shape, X-shape, hook shape, etc.) or a closed shape (e.g. circular or semi-circular shape, rectangular shape, triangular shape, etc.), and may be formed from the same material as the string (e.g., may be a loop or knot in the string) or may comprise different material (e.g., plastic, metal, etc.). Suitable handles are demonstrated herein, in particular in FIGS. 21, 22, 23, and 24.

In some embodiments, the handle also serves as means to store the ingestible device prior to use in a subject. For example, the handle may comprise a cavity in which the dissolvable capsule may be contained. The handle may also comprise a means to wind the string (e.g., suture) around the handle during storage.

In some embodiments, the handle pinches dissolvable capsule ingestible device within a set of grippers at one end of the handle. The handle may comprise a mechanism to loosen or unlock the grippers, thereby releasing the dissolvable capsule prior to use in a subject. In some embodiments, the other end of the handle (e.g. the end opposite to the grippers) is a hook. Such an embodiment is shown in FIG. 20.

In some embodiments, the handle comprises a flat surface containing a cavity in which the dissolvable capsule may be placed and a segment around which the suture may be wrapped. The handle may further comprise a means to release tension on the suture, thereby facilitating removal of the length of suture without the need for unwinding. Such an embodiment is shown, for example, in FIG. 21. In this particular figure, the handle comprises tabs, which may be squeezed to remove tension from the suture and allow for facile removal of the suture from the handle.

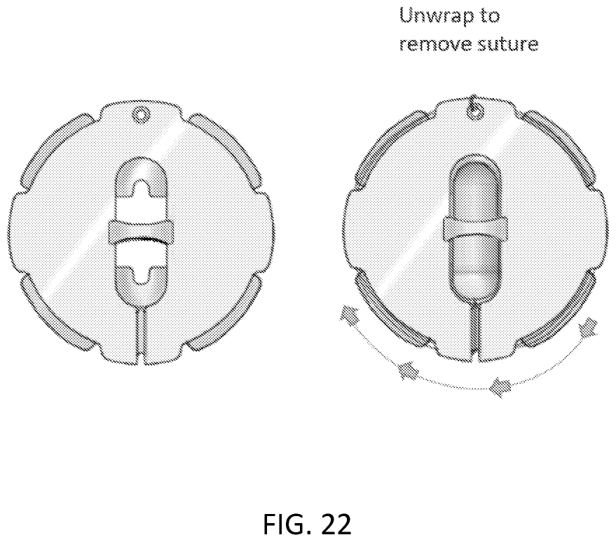

In some embodiments, the handle is circular in shape. The handle comprises a flat surface containing a cavity in which the dissolvable capsule may be placed. The suture may be wound around the external edge of the circular handle, such as along a slightly recessed channel extending along the outer edge of the handle. The suture may be tied in such as a position, which may be facilitated by a single hole placed in the circular handle. The suture may be untied and unwound from the circular handle to allow for the subject to ingest the device. Such an embodiment is shown in FIG. 22.

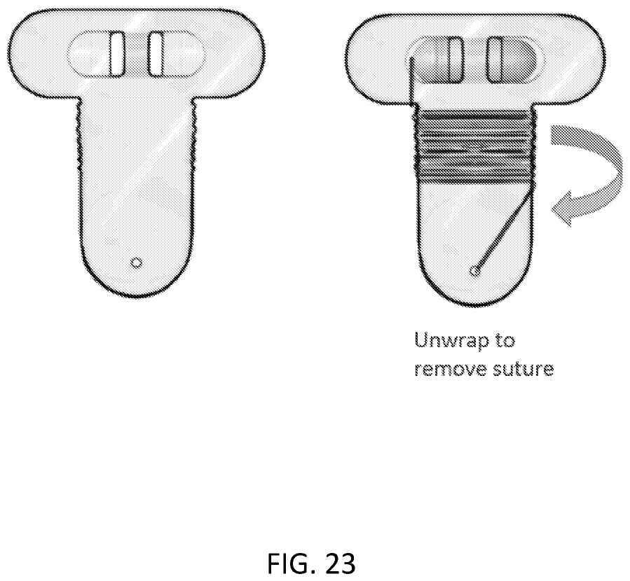

In some embodiments, the handle is a T-shape. The top cross section of the T may comprise a cavity in which the dissolvable capsule may be placed, whereas the perpendicular cross section may be used to wind the suture around the handle. Such an embodiment is shown, for example, in FIG. 23.

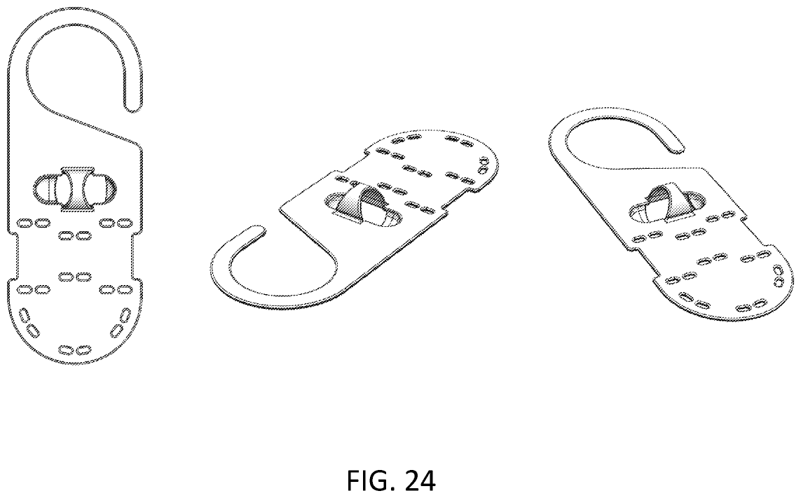

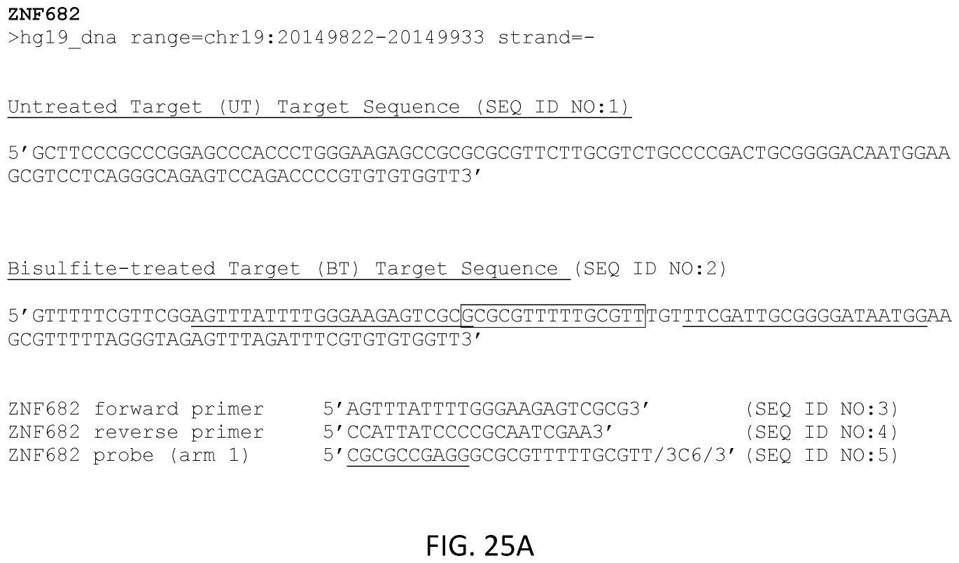

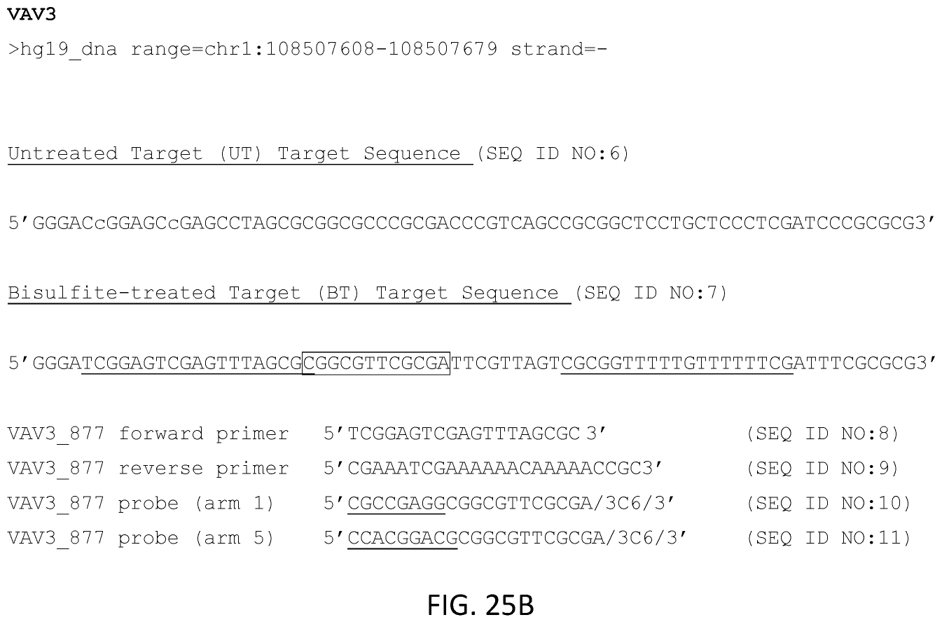

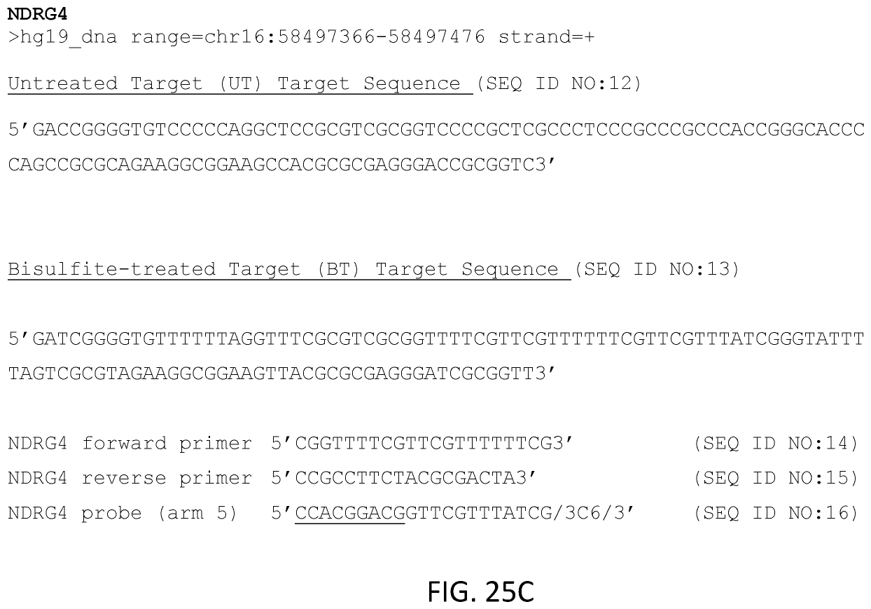

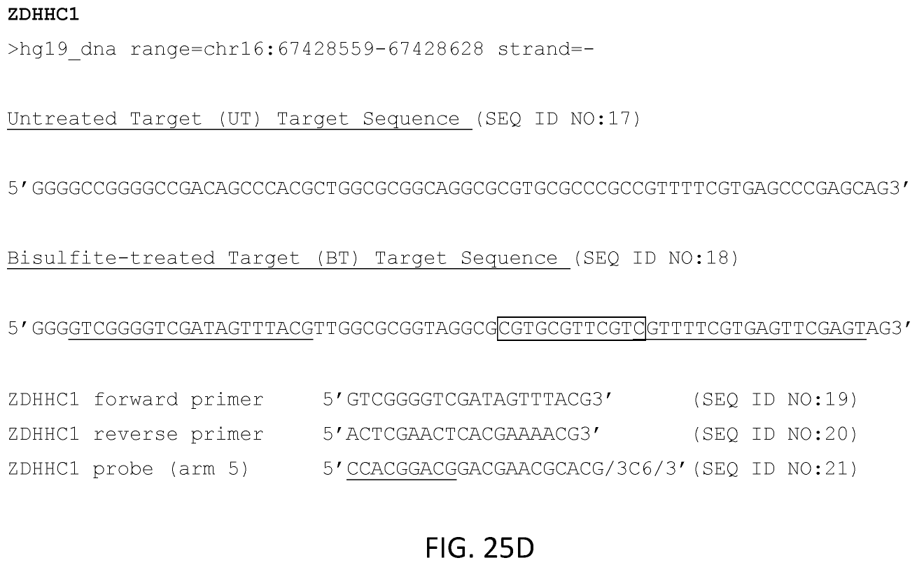

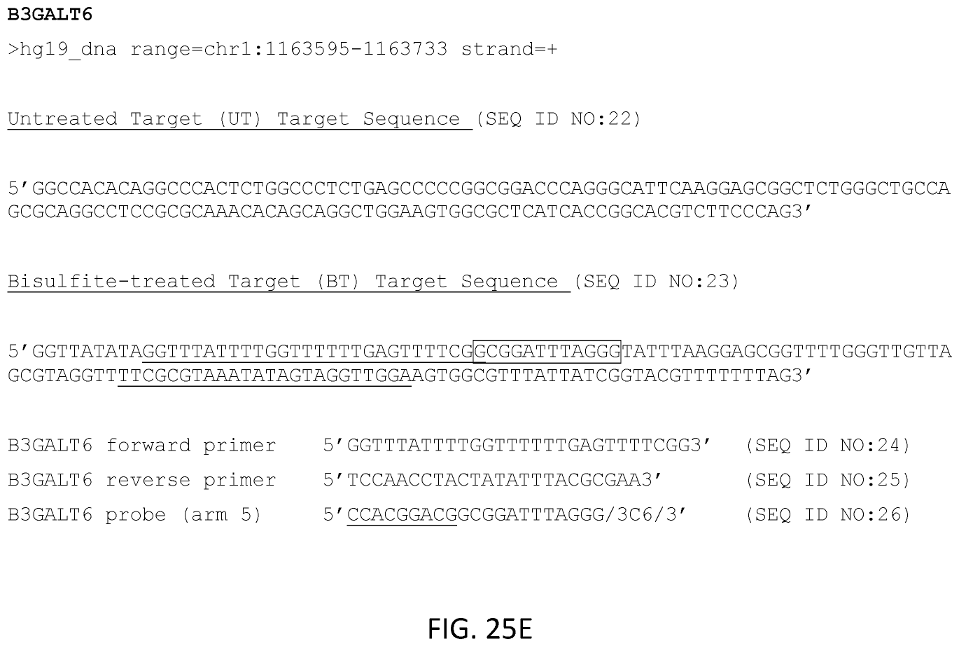

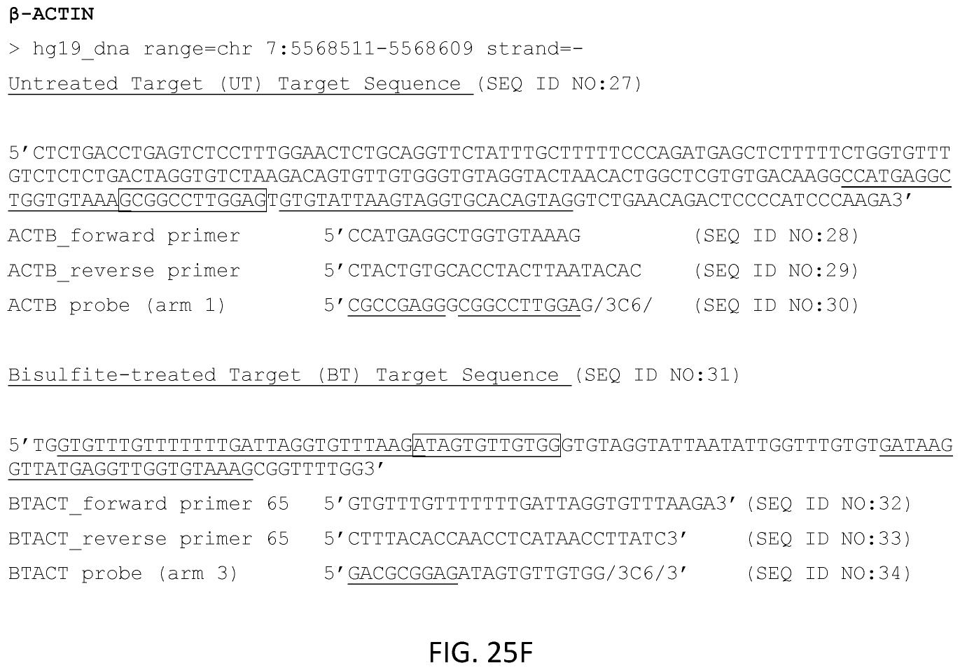

In some embodiments, the handle comprises a modified hook shape with multiple attachment sites to which the suture may be secured. FIG. 24 shows an exemplary embodiment of a handle as described herein. The handle comprises a flat surface on one end, and a hook shape on the opposing end. The flat surface comprises a cavity in which the dissolvable capsule may be placed. The flat surface additionally comprises a plurality of openings to provide a variety of suitable attachment sites for the suture.

In some embodiments, the string passes through a portion of the abrasive sponge. Accordingly, passing the string through the sponge will help secure the sponge to the molded cap, such that the sponge is not lost within the subject after dissolution of the dissolvable capsule. In some embodiments, the string passes through at least one surface of the dissolvable capsule. For example, the string may pass through the first closed end of the dissolvable capsule, through the abrasive sponge, and then attach to the molded cap. In some embodiments, the string may pass through the first closed end of the dissolvable capsule, through the abrasive sponge, through the second closed end of the dissolvable capsule, and then attach to the molded cap. The end of the string not attached to the molded cap may be attached to a handle, as described above.

Further described herein are methods for collecting cells from a subject. The methods comprise providing an ingestible cell sampling device described herein to the subject. Suitable methods for providing an ingestible cell sampling device to a subject are described in U.S. Pat. Nos. 4,735,214, 10,327,742, and 10,292,687, each of which are incorporated herein by reference in their entireties. For example, the subject may swallow the ingestible cell sampling device described herein and a suitable amount of time may pass prior to retrieving the device from the subject. For example, the subject may swallow the ingestible cell sampling device and 10 minutes or less may be allowed to pass prior to retrieval. For example, 10 minutes, 9 minutes, 8 minutes, 7 minutes, 6 minutes, 5 minutes, 4 minutes, 3 minutes, 2 minutes, or 1 minute may pass prior to retrieval. Retrieval may comprise having the subject, physician, or otherwise suitable person grab and pull on the string at a suitable rate to allow for comfortable retrieval of the device from the subject. Esophageal cells may be harvested from the abrasive sponge by any suitable means and subsequently analyzed to determine whether one or more abnormalities are present in the subject. In some embodiments, the esophageal cells may be harvested and placed in a suitable stabilization buffer prior to analysis. For example, a stabilization buffer may comprise any suitable agent or combination of agents that prevent unwanted damage to the cells (e.g. cell lysis) or damage/degradation of the nucleic acids (e.g. DNA or RNA) contained within the cell sample.

In some embodiments, esophageal cells are harvested from the abrasive sponge and analyzed to determine whether an esophageal disorder is present in the subject. Analysis may be performed by any suitable method, including protein-based tests, tissue/cell examinations (e.g., microscopy or other visual inspections), and/or nucleic acid detection assays. For example, analysis may be performed by protein-based techniques to analyze one or more biomarkers of interest. Protein-based techniques include, for example, immunohistochemistry, ELISA, western blot, flow cytometry, fluorescent in-situ hybridization (FISH), fluorescence analysis of cell sorting (FACS), mass spectrometry, etc. For example, protein-based techniques may be performed using one or more antibodies against at least one biomarker protein of interest. The biomarker protein(s) may be detected using an antibody capable of reacting with the protein(s), and subsequent visualization of the antibody. The antibody may be a polyclonal antibody or a monoclonal antibody. The use of secondary, tertiary or further antibodies may advantageously employed in order to amplify the signal and facilitate detection.

In some embodiments, esophageal cells are harvested from the abrasive sponge and examined to determine whether an esophageal disorder is present in the subject. For example, cells may be harvested from the sponge, plated on an appropriate medium, and examined by microscopy or other visual examination to determine whether characteristics indicative of an esophageal disorder are present in the cells. In some embodiments, cells may be harvested from the sponge, plated, and inspected using a microscope to determine whether one or more cancer cells are present. In some embodiments, diagnosis of an esophageal disorder may be made by visualization of a specific cell type, such as a columnar cell, which may be indicative of gastroesphageal reflux disease or complications thereof, including Barrett's Esophagus or esophageal adenocarcinoma.

In some embodiments, esophageal cells are harvested from the abrasive sponge and one or more nucleic acid detection assays are performed to determine whether an esophageal disorder is present in the subject. For example, esophageal cells may be harvested from the abrasive sponge following use in a subject, and the cells may be analyzed by one or more nucleic acid detection assays to detect levels of one or more biomarkers of an esophageal disorder. Suitable methods (e.g. nucleic acid detection assays) and biomarkers for detecting esophageal disorders are described in U.S. patent application Ser. No. 15/881,409 of Allawi, et al., filed Jan. 26, 2018, (including, e.g., ANKRD13B, CHST2, CNNM1, DOCK2, DTX1, FER1L4, FERMT3, FLI1, GRIN2D, JAM3, LRRC4, OPLAH, PDGFD, PKIA, PPP2R5C, QKI, SEP9, SFMBT2, SLC12A8, TBX15, TSPYL5, VAV3, ZNF304, ZNF568, and ZNF671), and U.S. Pat. No. 10,435,755, (including, e.g., BMP3, NDRG4, VAV3, SFMBT2, DIO3, HUNK, ELMO1, CD1D, CDKN2A; and OPLAH), both of which are incorporated herein by reference in their entireties. Exemplary assay designs for suitable biomarkers ZNF682, NDRG4, and VAV3 are discussed in more detail, below.

Exemplary nucleic acid assay designs are shown in FIG. 16A-16F. For example, esophageal cells may be harvested from the abrasive sponge and the levels of one or more biomarkers selected from ZNF682, NDRG4, and VAV3 may be determined. In some embodiments, levels of ZNF682, NDRG4, and VAV3 may be determined. In some embodiments, detecting an esophageal disorder may comprise measuring DNA methylation levels of the one or more biomarkers.

In some embodiments, the biomarker may be ZNF682. Exemplary primers and probes for ZNF682 are shown in FIG. 16A. In some embodiments, the ZNF682 forward primer may comprise 5′AGTTTATTTTGGGAAGAGTCGCG3′ (SEQ ID NO: 3), the reverse primer may comprise 5′CCATTATCCCCGCAATCGAA3′ (SEQ ID NO: 4), and the probe may comprise 5′CGCGCCGAGGGCGCGTTTTTGCGTT/3C6/3′(SEQ ID NO: 5).

In some embodiments, the biomarker may be VAV3. Exemplary primers and probes for VAV3 are shown in FIG. 16B. In some embodiments, the VAV3 forward primer may comprise 5′TCGGAGTCGAGTTTAGCGC3′ (SEQ ID NO: 8) and the reverse primer may comprise 5′CGAAATCGAAAAAACAAAAACCGC3′ (SEQ ID NO: 9). In some embodiments, VAV3 may be detected by one probe or two probes. For example, VAV3 may be detected by the probe (arm 1) 5′CGCCGAGGCGGCGTTCGCGA/3C6/3′ (SEQ ID NO: 10) and/or the probe (arm 5) 5′CCACGGACGCGGCGTTCGCGA/3C6/3′ (SEQ ID NO: 11).

In some embodiments, the biomarker may be NDRG4. Exemplary primers and probes for NDRG4 are shown in FIG. 16C. In some embodiments, the NDRG4 forward primer may comprise 5′CGGTTTTCGTTCGTTTTTTCG3′ (SEQ ID NO: 14), the reverse primer may comprise 5′CCGCCTTCTACGCGACTA3′ (SEQ ID NO: 15), and the probe may comprise 5′CCACGGACGGTTCGTTTATCG/3C6/3′ (SEQ ID NO: 16).

In some embodiments, the biomarker may be bone morphogenic protein 3 (BMP3). In some embodiments, the biomarker may be ZNF568. In some embodiments, the biomarker may be BMP3 and ZNF568.

In some embodiments, one or a group of biomarkers for analyzing a sample collected from an esophagus, e.g., for detecting esophageal disorders, may be selected from the group consisting of NDRG4, ZNF682, VAV3, BMP3, ZNF568, FER1L4, ANKRD13B, CD1D, CDKN2A , CHST2, CNNM1, DIO3, DOCK2, DTX1, ELMO1, FERMT3, FLI1, GRIN2D, HUNK , JAM3, LRRC4, OPLAH, PDGFD, PKIA, PPP2R5C, QKI, SEP9, SFMBT2, SLC12A8, TBX15, TSPYL5, ZNF304, and ZNF671. Biomarkers selected from this group may comprise 1 biomarker, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 biomarkers, alone or in any combination or subcombination, without limitation. For example, in certain embodiments, the biomarker or group of biomarkers is selected from the group consisting of ANKRD13B, CHST2, CNNM1, DOCK2, DTX1, FER1L4, FERMT3, FLI1, GRIN2D, JAM3, LRRC4, OPLAH, PDGFD, PKIA, PPP2R5C, QKI, SEP9, SFMBT2, SLC12A8, TBX15, TSPYL5, VAV3, ZNF304, ZNF568, and ZNF671, while in some embodiments, the biomarker or group of biomarkers is selected from the group consisting of BMP3, NDRG4, VAV3, SFMBT2, DIO3, HUNK, ELMO1, CD1D, CDKN2A, and OPLAH. In some embodiments, the biomarker or group of biomarkers is selected from the group consisting of NDRG4, ZNF682, VAV3, BMP3, ZNF568, and FER1L4, and in certain embodiments, the group of biomarkers comprises the group consisting of NDRG4, ZNF682, VAV3, BMP3, ZNF568, and FER1L4.

In some embodiments, the one or more biomarkers are normalized against a reference marker. Suitable methods and reference markers are described in U.S. Pat. No. 10,465,248 and U.S. patent application Ser. No. 16/318,580, the entire contents of each of which are incorporated herein by reference. In some embodiments, the reference marker is selected from β-actin, ZDHHC1, and B3GALT6.

In some embodiments, the reference marker may be ZDHHC1. Exemplary primers and probes for ZDHHC1 are shown in FIG. 16D. In some embodiments, the ZDHHC1 forward primer comprises 5′GTCGGGGTCGATAGTTTACG3′ (SEQ ID NO: 19), the reverse primer comprises 5′ACTCGAACTCACGAAAACG3′ (SEQ ID NO: 20), and the probe comprises 5′CCACGGACGGACGAACGCACG/3C6/3′ (SEQ ID NO: 21).

In some embodiments, the reference marker may be B3GALT6. Exemplary primers and probes for B3GALT6 are shown in FIG. 16E. In some embodiments, the B3GALT6 forward primer comprises 5′GGTTTATTTTGGTTTTTTGAGTTTTCGG3′ (SEQ ID NO:24), the reverse primer comprises 5′TCCAACCTACTATATTTACGCGAA3′ (SEQ ID NO:25), and the probe comprises 5′CCACGGACGGCGGATTTAGGG/3C6/3′ (SEQ ID NO:26).

In some embodiments, the reference marker may be β-actin. Exemplary primers and probes for β-actin are shown in FIG. 16F. In some embodiments, the β-actin forward primer comprises 5′GTGTTTGTTTTTTTGATTAGGTGTTTAAGA3′ (SEQ ID NO:32), the reverse primer comprises 5′CTTTACACCAACCTCATAACCTTATC3′ (SEQ ID NO:33), and the probe comprises 5′GACGCGGAGATAGTGTTGTGG/3C6/3′ (SEQ ID NO:34).

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

We claim:

1. An ingestible cell sampling device comprising:
i) an abrasive sponge housed within a dissolvable capsule, the dissolvable capsule comprising an exterior surface exposed to an external environment;
ii) a molded cap; and
iii) a string having a first end attached to the molded cap.

2. The ingestible cell sampling device of claim 1, further comprising a handle, preferably an unswallowable handle, attached to the string.

3. The ingestible cell sampling device of claim 1 or 2, wherein the abrasive sponge comprises reticulated foam.

4. The ingestible cell sampling device of any of the preceding claims, wherein the abrasive sponge is compressible.

5. The ingestible cell sampling device of claim 4, wherein the abrasive sponge is retained in a compressed state by the dissolvable capsule.

6. The ingestible cell sampling device of any of the preceding claims, wherein in an uncompressed state, the abrasive sponge comprises at least one void space.

7. The ingestible cell sampling device of claim 6, wherein the string passes through at least one void space, preferably at least one concavity.

8. The ingestible cell sampling device of any of the preceding claims, wherein the dissolvable capsule comprises one or more openings, wherein a portion of the abrasive sponge is exposed to the external environment at the one or more openings.

9. The ingestible cell sampling device of any of the preceding claims, wherein the dissolvable capsule comprises a first end and a second end, wherein:
a) the first end is closed and the second end is closed; or
b) the first end is closed and the second end is open.

10. The ingestible cell sampling device of claim 9, wherein the molded cap comprises a cap interior surface and a cap exterior surface, wherein the cap interior surface is in contact with the exterior surface of the capsule at the first closed end, and the cap exterior surface is in contact with the external environment.

11. The ingestible cell sampling device of claim 9, wherein the molded cap comprises a cap interior surface and a cap exterior surface, wherein the cap interior surface is in contact with the abrasive sponge.

12. The ingestible cell sampling device of claim 11, wherein the cap exterior surface is in contact with an internal surface of the capsule at the first closed end.

13. The ingestible cell sampling device of claim 11 or 12, wherein the cap interior surface is attached to the abrasive sponge by an adhesive.

14. The ingestible cell sampling device of claim 9, wherein the molded cap comprises a cap interior surface in contact with the abrasive sponge and a cap exterior surface in contact with the external environment.

15. The ingestible cell sampling device of claim 14, wherein the cap interior surface is attached to the abrasive sponge.

16. The ingestible cell sampling device of claim 15, wherein the cap interior surface is attached to the abrasive sponge by an adhesive.

17. The ingestible cell sampling device of any of the preceding claims, wherein the string is attached to the molded cap by a knot and/or an adhesive.

18. The ingestible cell sample device of any of the proceeding claims, wherein the string has one or more calibration markings.

19. The ingestible cell sampling device of any of the preceding claims, wherein the string comprises a suture.

20. The ingestible cell sampling device of any of the preceding claims, wherein the string passes through a portion of the abrasive sponge.

21. The ingestible cell sampling device of any of the preceding claims, wherein the molded cap comprises a button.

22. A system or kit for obtaining a cell sample from a subject, comprising an ingestible cell sampling device of any of the preceding claims; and further comprising one or more of:
i) a container to receive an abrasive sponge comprising collected cells;
ii) a cell preservative reagent, preferably a buffer reagent;
iii) a microscope slide;
iv) an assay plate;
v) a local anesthetic treatment, preferably a local anaesthetic spray;
vi) a component of a drinkable solution; preferably a pre-mixed drinkable solution; and
vii) a lubricant, preferably a lubricant gel or liquid.

23. A method of obtaining a cell sample from a subject, comprising:
i) orally administering an abrasive sponge housed within a dissolvable capsule of a ingestible cell sampling device of any one of claims 1-21 to the subject, and
ii) withdrawing from the subject the abrasive sponge, wherein the abrasive sponge collects a cell sample from the subject during the withdrawing.

24. The method of claim 23, wherein the withdrawing is within 10 minutes of the orally administering.

25. The method of claim 23 or claim 24, wherein during the orally administering, the subject swallows the dissolvable capsule of the ingestible cell sampling device.

26. A method of characterizing a cell sample collected according to any one of claims 23-25, comprising assaying the cell sample for at least one biomarker.

27. The method of claim 26, wherein the at least one biomarker is selected comprises one or more of a protein and a nucleic acid.

28. The method of claim 26 or claim 27, wherein the at least one biomarker comprises DNA comprising at least a portion of a gene selected from the group consisting of NDRG4, ZNF682, VAV3, BMP3, ZNF568, FER1L4, ANKRD13B, CD1D, CDKN2A , CHST2, CNNM1, DIO3, DOCK2, DTX1, ELMO1, FERMT3, FLI1, GRIN2D, HUNK, JAM3, LRRC4, OPLAH, PDGFD, PKIA, PPP2R5C, QKI, SEP9, SFMBT2, SLC12A8, TBX15, TSPYL5, ZNF304, and ZNF671.

29. The method of claim 28, wherein assaying the at least one biomarker comprises determining the DNA to determine the methylation state of the gene.

30. The method of any one of claims 26-29, wherein assaying the at least one biomarker comprises assaying 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 of the biomarkers from the group consisting of NDRG4, ZNF682, VAV3, BMP3, ZNF568, FER1L4, ANKRD13B, CD1D, CDKN2A , CHST2, CNNM1, DIO3, DOCK2, DTX1, ELMO1, FERMT3, FLI1, GRIN2D, HUNK, JAM3, LRRC4, OPLAH, PDGFD, PKIA, PPP2R5C, QKI, SEP9, SFMBT2, SLC12A8, TBX15, TSPYL5, ZNF304, and ZNF671.

31. The method of claim 30, wherein assaying the at least one biomarker comprises assaying the methylation state of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 genes from the group consisting of NDRG4, ZNF682, VAV3, BMP3, ZNF568, FER1L4, ANKRD13B, CD1D, CDKN2A , CHST2, CNNM1, DIO3, DOCK2, DTX1, ELMO1, FERMT3, FLI1, GRIN2D, HUNK, JAM3, LRRC4, OPLAH, PDGFD, PK1A, PPP2R5C, QKI, SEP9, SFMBT2, SLC12A8, TBX15, TSPYL5, ZNF304, and ZNF671.

31. The method of any one of claims 28-30, wherein assaying the at least one biomarker comprises assaying the methylation state of at least one gene selected from the group consisting of: ANKRD13B, CHST2, CNNM1, DOCK2, DTX1, FER1L4, FERMT3, FLI1, GRIN2D, JAM3, LRRC4, OPLAH, PDGFD, PK1A, PPP2R5C, QKI, SEP9, SFMBT2, SLC12A8, TBX15, TSPYL5, VAV3, ZNF304, ZNF568, and ZNF671.

32. The method of any one of claims 28-30, wherein assaying the at least one biomarker comprises assaying the methylation state of at least one gene selected from the group consisting of: BMP3, NDRG4, VAV3, SFMBT2, DIO3, HUNK, ELMO1, CD1D, CDKN2A, and OPLAH.

33. The method of any one of claims 28-30, wherein assaying the at least one biomarker comprises assaying the methylation state of at least one gene selected from the group consisting of NDRG4, ZNF682, VAV3, BMP3, ZNF568, and FER1L4.

34. The method of claim 33, comprising assaying the methylation state of the group of genes consisting of NDRG4, ZNF682, VAV3, BMP3, ZNF568, and FER1L4.
